SEQUENCE LISTING (26 sequences)

SEQ ID NO: 1 - DNA, 1731 bp, Campanula medium
  CDS (2)..(940): F3′5′H derived from Campanula genome DNA
catgtctatagacatatccaccctcttctatgaacttgttgcagcaatttcactctacttagctacctactctttcattcgtttcctcttcaaaccctctcaccaccaccacctccctcccggcccaaccggatggccgatcatcggagcccttccactcttaggcaccatgccacatgtttccttagccgacatggccgttaaatacggtcctataatgtacctaaaacttggttcaaagggcaccgtcgtggcctcaaatccaaaagccgcccgagccttcttgaaaacccatgatgccaatttttctaaccgtccgattgatgggggccctacctacctcgcgtataatgcacaagacatggtttttgcagaatatggcccaaaatggaagcttttgcgaaagctatgtagcttgcacatgttaggcccgaaggcactcgaggattgggctcatgtcaaagtttcagaggtcggtcatatgctcaaagaaatgtacgagcaatcgagtaagtcagtgccagtgccagtggtggtgccagagatgttaacttatgccatggctaatatgattggacgaatcatactcagccgacgcccttttgttatcacgagcaaattagactcgtctgcttctgcttctgcttctgttagtgaattccaatatatggttatggagctcatgaggatggcagggttgttcaatattggtgatttcataccatatattgcatggatggatttgcaaggcattcagcgtgatatgaaggttatacagaaaaagtttgatgtcttgttgaacaaaatgatcaaggaacatacagaatccgctcatgatcgcaaagataatcctgattttcttgatattcttatggcggctacccaagaaaacacggagggaattcagcttaatcttgtaaatgttaaggcacttcttttggtacgtatcatcaacttggatttgtatgaacatctaacatttttctggaaaaagagctaaaaagggcactctttaaagggtacttatttgtgacgaatattatgcttgaaaatgaaatgaatacttgttgaaattaatgctgtatatttatgctaggatttattcacggcgggcacggatacatcatcaagtgtgatcgaatgggcactagccgaaatgttgaaccatcgacagatcctaaaccgggcccacgaagaaatggaccaagtcattggcagaaacagaagactagaacaatctgacataccaaacttgccatatttccaagccatatgcaaagaaacattccgaaaacacccttccacgcccttaaacctcccaagaatctcaacagaagcatgtgaagtggacggatttcacataccaaaaaacactagactaatagtgaacatatgggcaatagggagggaccctaaagtgtgggaaaatccattagattttactccggaacgtttcttgagtgaaaaacacgcgaaaattgacccgcgaggtaatcattttgagttaatcccatttggggctggacgaaggatatgtgcaggggctagaatgggagcggcctcggtcgagtacatattaggtacattggtgcactcatttgattggaaattgcctgatggagttgtggaagttaatatggaagagagctttgggatcgcattgcaaaaaaaggtgcctctttctgctattgttactccaagattgcctccaagttcttacactgtctaggc

SEQ ID NO: 2 - protein, 523 aa, Campanula medium
MSIDISTLFYELVAAISLYLATYSFIRFLFKPSHHHHLPPGPTGWPIIGALPLLGTMPHVSLADMAVKYGPIMYLKLGSKGTVVASNPKAARAFLKTHDANFSNRPIDGGPTYLAYNAQDMVFAEYGPKWKLLRKLCSLHMLGPKALEDWAHVKVSEVGHMLKEMYEQSSKSVPVPVVVPEMLTYAMANMIGRIILSRRPFVITSKLDSSASASASVSEFQYMVMELMRMAGLFNIGDFIPYIAWMDLQGIQRDMKVIQKKFDVLLNKMIKEHTESAHDRKDNPDFLDILMAATQENTEGIQLNLVNVKALLLDLFTAGTDTSSSVIEWALAEMLNHRQILNRAHEEMDQVIGRNRRLEQSDIPNLPYFQAICKETFRKHPSTPLNLPRISTEACEVDGFHIPKNTRLIVNIWAIGRDPKVWENPLDFTPERFLSEKHAKIDPRGNHFELIPFGAGRRICAGARMGAASVEYILGTLVHSFDWKLPDGVVEVNMEESFGIALQKKVPLSAIVTPRLPPSSYTV

SEQ ID NO: 3 - DNA, 575 bp, Lilium sp.
  CDS (76)..(573): cDNA of Lily F3′H
ttggctggtgtttttaacataggtgattttgtgccggggctgggttggttggatttgcagggggtggtggggaagatgaagaagttgcataagaggatggatgcttttcttgataagttgatagcggagcaccggacggcagcggccggcggcgagttcgccggaaaagatttgttgagtgtgatggttaggttgcaggaggagggagagggggagggtgggaaactgaccgacactgaaattaaggcgctgttactgaacttgttcacagcagggacagacaccacctccagcaccgtagagtgggctctggccgagctcatccgtcaccctccactcctccaccaagctcagaaagagcttgatactatcgtcggtcacgaccgtctcgtctctgaatccgacctcccccacctccccttcctaagtgccatcatcaaagaaactttccgactccacccatccaccccactctcactcccacggatggcctccgaatcatgcaccatcaatggctaccaaattcctaagaacgccactctactggtaaatgtgtgggccatagcccgtgaccc

SEQ ID NO: 4 - protein, 166 aa, Lilium sp.
MKKLHKRMDAFLDKLIAEHRTAAAGGEFAGKDLLSVMVRLQEEGEGEGGKLTDTEIKALLLNLFTAGTDTTSSTVEWALAELIRHPPLLHQAQKELDTIVGHDRLVSESDLPHLPFLSAIIKETFRLHPSTPLSLPRMASESCTINGYQIPKNATLLVNVWAIARD

SEQ ID NO: 5 - DNA, 35 bp, Artificial Sequence
  Description of Artificial Sequence Synthetic oligonucleotide CFH1L
catgtctatagacatatccaccctcttctatgaac

SEQ ID NO: 6 - DNA, 35 bp, Artificial Sequence
  Description of Artificial Sequence Synthetic oligonucleotide cCF1L
gcctagacagtgtaagaacttggaggcaatcttgg

SEQ ID NO: 7 - DNA, 23 bp, Artificial Sequence
  Description of Artificial Sequence Synthetic oligonucleotide F3H2
ytsgcyggwgtwttyaacrthgg

SEQ ID NO: 8 - DNA, 20 bp, Artificial Sequence
  Description of Artificial Sequence Synthetic oligonucleotide cF3H4
ggrtcncgrgcwatggccca

SEQ ID NO: 9 - DNA, 21 bp, Artificial Sequence
  Description of Artificial Sequence Synthetic primer CamF1
gtgaagccaccatgtctatag

SEQ ID NO: 10 - DNA, 22 bp, Artificial Sequence
  Description of Artificial Sequence Synthetic primer CamR1
gcatttgcctagacagtgtaag

SEQ ID NO: 11 - DNA, 1585 bp, Campanula medium
  CDS (12)..(1574)
gtgaagccaccatgtctatagacataaccattctcttatgtgaacttgttgctgcaatttcactctacttattaacctactatttcatttgtttcctcttcaaaccctctcatcatcaccaccacctccctcccggcccaaccggatggccgatcattggatcccttcctctcttaggcactatgccacatgtttccttagccgacatggccgtaaaatacgggcctataatgtacctaaaacttggttcaaagggcaccgtcgtggcctcaaatccaaaagccgcccgagcattcttgaaatcccatgatgccaatttttctaaccgtccgattgatggggggcccacctacctcgcgtataatgcacaagacatggtttttgcagaatatggcccaaaatggaagcttttgcgaaagctatgtagcttgcacatgttaggcccgaaggcactcgaggattgggctcatgtcagagtttcagaggtcggtcatatgctcaaagaaatgtacgagcaatcgagtaagtccgtgccagtggtggtgccagagatgttaacttatgccatggctaatatgattggacgaatcatactcagtcgacgcccttttgttatcacgagcaaattagactcgtctgcttctgctgcttctgttagtgaattccaatatatggttatggagctcatgaggatggcagggttgttcaatattggtgatttcataccatatattgcgtggatggatttgcaaggcattcaacgcgatatgaaggttatacagcaaaagtttgatgtcttgttgaacaaaatgatcaaggaacatacagaatccgctcatgatcgtaaagataatcctgattttcttgatattcttatggcggctacccaagaaaacacggagggaattcagcttaatctcgtaaatgttaaggcgcttcttttggatttattcacggcgggcacggatacatcgtcgagtgtgatcgaatgggcactagccgaaatgttgaacaatcgacagatcctaaaccgggcccacgaagaaatggaccaagtcattggcagaaacagaagactagaacaatctgacataccaaacttgccatatttccaagccatatgcaaagaaacattccgaaaacacccttccacgcccttaaacctcccaagaatctcaacagaagaatgtgaagtcgaaggatttcgcatacccaaaaacactagactaatagtgaacatatgggcaatagggagagaccctaaagtgtgggaaaatccattggattttaccccggaacgattcttgagtgaaaaacacgcgaaaattgatccgcgaggtaatcattttgagttaatcccatttggggcgggacggaggatatgtgcaggggctagaatgggagcggcctcggtcgagtacattttaggtacattggtgcactcatttgattggaaattgcctgatggagttgtggaagttaatatggaagagagctttgggatagcattgcagaaaaagatgcctctttctgctattgttactccaagattgcctccaagtgcttacactgtctaggcaaatgc

SEQ ID NO: 12 - protein, 521 aa, Campanula medium
MSIDITILLCELVAAISLYLLTYYFICFLFKPSHHHHHLPPGPTGWPIIGSLPLLGTMPHVSLADMAVKYGPIMYLKLGSKGTVVASNPKAARAFLKSHDANFSNRPIDGGPTYLAYNAQDMVFAEYGPKWKLLRKLCSLHMLGPKALEDWAHVRVSEVGHMLKEMYEQSSKSVPVVVPEMLTYAMANMIGRIILSRRPFVITSKLDSSASAASVSEFQYMVMELMRMAGLFNIGDFIPYIAWMDLQGIQRDMKVIQQKFDVLLNKMIKEHTESAHDRKDNPDFLDILMAATQENTEGIQLNLVNVKALLLDLFTAGTDTSSSVIEWALAEMLNNRQILNRAHEEMDQVIGRNRRLEQSDIPNLPYFQAICKETFRKHPSTPLNLPRISTEECEVEGFRIPKNTRLIVNIWAIGRDPKVWENPLDFTPERFLSEKHAKIDPRGNHFELIPFGAGRRICAGARMGAASVEYILGTLVHSFDWKLPDGVVEVNMEESFGIALQKKMPLSAIVTPRLPPSAYTV

SEQ ID NO: 13 - DNA, 1322 bp, Lilium sp.
  CDS (1)..(1158): cDNA of lily DFR
atgtctcaaagaggagagagagagatggagaatgtggtgaaaggccccgtggtggtgactggtgctagtggctatgttggttcatggcttgtaatgaagcttctgcaatatggatacacggtccgggctactgtgagagaccctagagacctaagaaagaccaaacctttaatagatcttcctggagctgatgaacggctaaccatttggaaagcagatcttagcgaggatgagagctttgatgaggcaatcaatggttgctccggtgtgtaccatgttgcaactccgatggatttcgattccgaagatccggagaatgaagtgatcaagcccacaataaatggagtacttggtatcatgaaatcttgcaaaaaagcagggacggtgaagcgtgtcattttcacttcctctgctggaactgtgaacgtgcaagaacatcaaatgtccgaatatgacgagagctcgtggagcgacatcgacttctgcagacgcgtcaagatgactggatggatgtactttgtatctaaaaccctagcagagaaagctgcatgggattttgcaaaggagaataatattcattttataagcattattccaactttggtggtgggtcctttcatcacctcaactatgcctcccagtatgctcacagctttgtcattgatcacaggaaatgaagcccactattcgatcttaaaacaaatccagcttgttcacttggatgacgtatgcaaggcccatattttcctctttgagaatccagaagcgagtgggagatacatatgctcttcttatgatgctacaatttacgatcttgcaagaaagattaaagatagatatcctcaatatgccatcccacaaaagtttgaaggtattgatgaccaaattaaacctgtgcacttctcttcaaagaaacttatggatcttggtttcaagtatcagtacacttttgaggagatgttcgatgaaggaatccgttcatgtatcgaaaagaaactgataccacaccaaacacaagagagatactatgctaatgataaactgaatctgggttacactaaaatgactaatgataaactggatttgggtggcataaagttgaattcaatggacgagattgtgaaggggcacaatgaacaagtctccgttgctcttcagtaagcttcggaggaccatacgaaagtaagccagactatgttgtagtaataactatagttgtgatttatgattcatatgtatgtgtgaggcactgagctgagactatgtggtagtaataactatagttgtgatttatgattcaaaaaaaaaaaaaaaaaaaaaaa

SEQ ID NO: 14 - protein, 386 aa, Lilium sp.
MSQRGEREMENVVKGPVVVTGASGYVGSWLVMKLLQYGYTVRATVRDPRDLRKTKPLIDLPGADERLTIWKADLSEDESFDEAINGCSGVYHVATPMDFDSEDPENEVIKPTINGVLGIMKSCKKAGTVKRVIFTSSAGTVNVQEHQMSEYDESSWSDIDFCRRVKMTGWMYFVSKTLAEKAAWDFAKENNIHFISIIPTLVVGPFITSTMPPSMLTALSLITGNEAHYSILKQIQLVHLDDVCKAHIFLFENPEASGRYICSSYDATIYDLARKIKDRYPQYAIPQKFEGIDDQIKPVHFSSKKLMDLGFKYQYTFEEMFDEGIRSCIEKKLIPHQTQERYYANDKLNLGYTKMTNDKLDLGGIKLNSMDEIVKGHNEQVSVALQ

SEQ ID NO: 15 - DNA, 20 bp, Artificial Sequence
  Description of Artificial Sequence Synthetic oligonucleotide LiRG-R3
tgctccgctatcaacttatc

SEQ ID NO: 16 - DNA, 1673 bp, Lilium sp.
  CDS (22)..(1563): full length of cDNA of lily F3′H
aaaaccaaaccgtcagccaccatggaagctcaacctctcctcctcctcctcaccaccatcctcatctccaccatcttcatcttcctccaccgccgacgccgcggcggcctaccactccctcctggcccgaaaggctggcctatccttggcaaccttccccaactcggccccaaaccccaccaaaccatgcacgccctatccaaactccacggccccctcttccgtctccgcctcggctctgccgaggtcatcgtcgccgcctcagcatcgatagcatctgagtttctccgcacgcacgacacaaacttcagcaatcgtcccccgaattctggtgccgaacacatagcttacaactacaatgaccttgtctttgccccctacggcccgcgatggcgtgctctccgcaagctttgcgccctccacctcttttcccagaaagcactcgaagatctctcctacgtccgcgagcaagaagtggcaatactcgcacgagacttggccggctcatgccggccggttaacctcggggagacacttaacgtttgtgcgacgaatgcgttgtcgagggcgacgatcgggtggagggtgtttgtggggaagggggaggatgtaacggccgaggattttaaggacatggtgatggaggtgatgcggctcgccggagtttttaatattggtgattttgtgccggggctgggttggttggatttgcagggggtggtggggaagatgaagaagttgcataagaggatggatgcttttcttgataagttgatagcggagcaccggacggcagcggccggcggcgagttcgccggaaaagatttgttgagtgtgatggttaggttgcaggaggagggagagggggagggtgggaaactgaccgacactgaaattaaggcgctgttactgaacttgttcacagcagggacagacaccacctccagcaccgtagagtgggctctggccgagctcatccgtcaccctccactcctccaccaagctcagaaagagcttgatactatcgtcggtcacgaccgtctcgtctctgaatccgacctcccccacctccccttcctaagtgccatcatcaaagaaactttccgactccacccatccaccccactctcactcccacggatggcctccgaatcatgcaccatcaatggctaccaaattcctaaaaacgccactctactggtaaatgtgtgggctatcgcacgtgacccagctgtttggacggccccggtagagttcaaaccggacaggttcatgccgggtggggttggtgctcacttggatgtaaagggtagcgacttcgagatgatcccttttggagccgggaggaggatttgcgctggtatgagcttggggctgaggatggtgacgttcatgacagcgacactagtgcatgggttcgaatggaagctcccgaatggggtggtggctgagaagttggacatggaggaggcttacggtctgaccctacagagggcggtaccgctgatggtggtgccagtgccgaggctcgcgaagcaggcgtatgggaagaatgagaaactctagtgtttgcatcttaataagaaaaacttattcgccatgattatcatgttataataatgacacttagaattaataaaaataaatttaaaatcaaaaaaaaaaaaaaaaaa

SEQ ID NO: 17 - protein, 514 aa, Lilium sp.
MEAQPLLLLLTTILISTIFIFLHRRRRGGLPLPPGPKGWPILGNLPQLGPKPHQTMHALSKLHGPLFRLRLGSAEVIVAASASIASEFLRTHDTNFSNRPPNSGAEHIAYNYNDLVFAPYGPRWRALRKLCALHLFSQKALEDLSYVREQEVAILARDLAGSCRPVNLGETLNVCATNALSRATIGWRVFVGKGEDVTAEDFKDMVMEVMRLAGVFNIGDFVPGLGWLDLQGVVGKMKKLHKRMDAFLDKLIAEHRTAAAGGEFAGKDLLSVMVRLQEEGEGEGGKLTDTEIKALLLNLFTAGTDTTSSTVEWALAELIRHPPLLHQAQKELDTIVGHDRLVSESDLPHLPFLSAIIKETFRLHPSTPLSLPRMASESCTINGYQIPKNATLLVNVWAIARDPAVWTAPVEFKPDRFMPGGVGAHLDVKGSDFEMIPFGAGRRICAGMSLGLRMVTFMTATLVHGFEWKLPNGVVAEKLDMEEAYGLTLQRAVPLMVVPVPRLAKQAYGKNEKL

SEQ ID NO: 18 - DNA, 34 bp, Artificial Sequence
  Description of Artificial Sequence Synthetic primer cLD2L
agggtctctcacagtagcccgaaccgtgtagcca

SEQ ID NO: 19 - DNA, 34 bp, Artificial Sequence
  Description of Artificial Sequence Synthetic primer LD1L
actagatcttcctggagctgatgaacggctaacc

SEQ ID NO: 20 - DNA, 35 bp, Artificial Sequence
  Description of Artificial Sequence Synthetic primer cLD1L
aacatagccactagctccagtaaccaccacgggtc

SEQ ID NO: 21 - DNA, 35 bp, Artificial Sequence
  Description of Artificial Sequence Synthetic primer LD3L
agcaaatccagcttgttcacttggatgacgtatgc

SEQ ID NO: 22 - DNA, 36 bp, Artificial Sequence
  Description of Artificial Sequence Synthetic oligonucleotide AscI-HindIII-LDE6-F
ggcgcgccaagcttgaattcttagcggaaattatcc

SEQ ID NO: 23 - DNA, 25 bp, Artificial Sequence
  Description of Artificial Sequence Synthetic oligonucleotide XbaI-LDE6-R
tctagaatctctctctctccccctc

SEQ ID NO: 24 - DNA, 613 bp, Lilium sp.
  promoter (23)..(590): lily DFR promoter
gaattcttagcggaaattatccggatccgtccgaaacataagataatccccgaacctgatcttttcgagatttggactctggacttctgtcaagaatgcgatttcagatacagtattactcccatccaatttgcttgtagagattttgatcttttatgtgttttgcataagtcttcctttaatgaggcttccttccttgaaaatattcctaccaatcaacatatgacgcggccgtctgtgctcctctcatggtacgagaaacatgggttcttttaagtgaggaaaacgaaaacggtaggccagagtccaattccaacaaccaatcaaaccagttgcatgtcggaacgtggttcattacccagttaaccttctaccattctctaggctgaaagttagataccaagttccattgtgctacgtaccacactcaatctaaaataagagattaatatacatataacagctgaaaattaaatacccactttccattttgccacctaccacgctcaacctaacatgacagattcatatatctagcatccgagattcattatataacagtcctacgtttcaaagagaagagagagagagggggagagagagagattctaga

SEQ ID NO: 25 - protein, 8 aa, Arabidopsis thaliana
LAGVFNIG

SEQ ID NO: 26 - protein, 9 aa, Arabidopsis thaliana
NIWAIARDP

(19) United States
(12) Patent Application Publication
(10) Pub. No.: US 2013/0347143 A1
(43) Pub. Date: Dec. 26, 2013

(54) METHOD FOR PRODUCING LILIES CONTAINING DELPHINIDIN IN THE PETALS THEREOF

(71) Applicants: Yoshikazu Tanaka, Mishima-gun (JP); Noriko Nakamura, Mishima-gun (JP); Hitoshi Kobayashi, Nagaoka-shi (JP); Hiroaki Okuhara, Nagaoka-shi (JP); Masayoshi Kondo, Nagaoka-shi (JP); Yosuke Koike, Nagaoka-shi (JP); Yosuke Hoshi, Uonuma-shi (JP); Toshikazu Nomizu, Kitakamahara-gun (JP)

(72) Inventors: Yoshikazu Tanaka, Mishima-gun (JP); Noriko Nakamura, Mishima-gun (JP); Hitoshi Kobayashi, Nagaoka-shi (JP); Hiroaki Okuhara, Nagaoka-shi (JP); Masayoshi Kondo, Nagaoka-shi (JP); Yosuke Koike, Nagaoka-shi (JP); Yosuke Hoshi, Uonuma-shi (JP); Toshikazu Nomizu, Kitakamahara-gun (JP)

(73) Assignees: NIIGATA PREFECTURE, Niigata-shi, Niigata (JP); SUNTORY HOLDINGS LIMITED, Osaka-shi, Osaka (JP)

(21) Appl. No.: 13/822,131

(22) PCT Filed: Sep. 16, 2011
(86) PCT No.: PCT/JP2011/071271

§ 371 Date: Mar. 11, 2013

(30) Foreign Application Priority Data
Sep. 17, 2010 (JP) 2010-209349

(51) Int. Cl. C12N 15/82

(52) U.S. Cl. CPC C12N 15/825
USPC 800/282; 800/321; 435/419

(57) ABSTRACT

Provided is a method for producing lilies containing a blue pigment (delphinidin) in the petals thereof by introducing a foreign gene into lilies. The method pertaining to the present invention is a method for producing lilies containing delphinidin in the petals thereof, including the following steps: introducing, into a lily, a F3′5′H gene derived from a campanula and comprising a nucleotide sequence encoding a peptide having flavonoid 3′5′-hydroxylase (F3′5′H) activity, such as a nucleotide sequence represented by SEQ ID NO: 1 or SEQ ID NO: 11; while also introducing a F3′H gene fragment derived from a lily and comprising a nucleotide sequence encoding a peptide having flavonoid 3′-hydroxylase activity, such as a nucleotide sequence represented by SEQ ID NO: 3 or SEQ ID NO: 16; and inhibiting the expression of endogenous F3′H expression, which acts on cyanidin synthesis in lily petals, while the F3′5′H gene that has been introduced acts to cause the synthesis of delphinidin.

### TECHNICAL FIELD

The present invention relates to a method for producing lilies containing a blue pigment (delphinidin) in the petals thereof by introducing a foreign gene into lilies. More particularly, the present invention relates to a method for producing lilies containing a blue pigment in the petals thereof by introducing flavonoid 3′,5′-hydroxylase (F3′5′H) gene derived from campanula required for delphinidin synthesis into a lily, and introducing a fragment of lily flavonoid 3′-hydroxylase (F3′H) gene in order to suppress expression of endogenous F3′H that acts on synthesis of red pigment (cyanidin), and inducing synthesis of delphinidin in lily petals, and to a lily produced by this method.

### BACKGROUND ART

Methods have been previously reported for inducing the production of delphinidin in plant cells by introducing a foreign gene in carnations (see Patent Document 1), chrysanthemums (see Patent Document 1), roses (see Patent Document 1 and Patent Document 2), moth orchids (see Patent Document 3) and cymbidium (see Patent Document 3).

However, cases of successfully producing lilies having blue petals by synthesizing delphinidin in lily petals using the action of an introduced foreign gene have yet to be reported.

Examples of methods used to improve plant varieties include (1) crossbreeding by crossing stamen and pistil, (2) spontaneous or artificial mutation, and (3) genetic recombination. The use of genetic recombination technology enables expression a useful gene in a plant without being constrained by genetic restrictions possessed by the target species, thereby making it possible to impart a new trait to a plant that is not inherently possessed by that plant. Genetically modified plants produced by genetic recombination technology are currently cultivated over a wide range throughout the world.

Examples of plant pigment components include anthocyanin, carotenoid and betalain.

Anthocyanins are members of the group of secondary metabolites referred to as flavonoids, and as shown in FIG. 1, are synthesized by the actions of numerous enzymes extending from phenylalanine to coumaroyl-CoA. The color of anthocyanins is dependent on the structure thereof. Namely, as the number of hydroxyl groups of the B ring of anthocyanidins, which is the chromophore of anthocyanins, increases, blue color becomes more intense, and major anthocyanidins in the form of delphinidin, cyanidin and pelargonidin have increasing numbers of hydroxyl groups in that order.

Anthocyanins derived from delphinidin accumulate in many blue flowers. In addition, as the number of aromatic acyl groups (such as coumaroyl groups or caffeoyl groups) that modify anthocyanins increases, the color of anthocyanins is known to become increasingly blue (namely, maximum absorbance shifts to a longer wavelength), and anthocyanin stability is known to increase. In particular, anthocyanins in which multiple aromatic acyl groups are bonded are referred to as acylated anthocyanins, and are contained in the blue petals of gentians and butterfly peas (see Non-Patent Document 1).

Enzymes involved in anthocyanin biosynthesis and genes that encode those enzymes are previously been isolated from numerous plants (see Non-Patent Document 1). The color of anthocyanins is dependent on such factors as the pH of vacuoles in which they are localized, other flavonoids and metal ions. Namely, anthocyanins appear red if vacuole pH is low, and become blue as vacuole pH becomes neutral. In addition, flavones and flavonols are referred to as co-pigments, and when present with anthocyanins, have the effect of causing the anthocyanins to appear blue. Moreover, iron and aluminum ions are known to cause anthocyanins to become blue by coordinating with hydroxyl groups of the B ring of anthocyanins.

Although plants produce flowers of various colors, there are only a few species capable of producing flowers of all colors. This is because pigments that can be synthesized by a species are determined genetically. For example, there are no blue or violet varieties of roses, carnations, chrysanthemums, lilies and gerbera. The main reason for this is that these plants do not have the flavonoid 3′,5′-hydroxylase (F3′5′H) gene required to synthesize delphinidin. Therefore, several attempts to produce blue flowers by inducing the production of delphinidin by expressing F3′5′H gene have been reported.

When F3′5′H gene is made to be expressed in petunias lacking F3′5′H, the amount of anthocyanins derived from delphinidin increases (see Non-Patent Document 2 and Non-Patent Document 3). In addition, when F3′5′H gene is expressed in tobacco (Nicotiana tabacum) that accumulates cyanidin, delphinidin is synthesized and flowers take on a somewhat bluish tint (see Non-Patent Document 3). In the case of having expressed F3′5′H gene derived from campanula, eustoma or petunia in tobacco, F3′5′H derived from campanula produced delphinidin with the greatest efficiency (see Non-Patent Document 4). In addition, in the case of having expressed the F3′5′H gene of butterfly pea or verbena in verbena, the case of having expressed the gene derived from butterfly pea yielded the larger production volume, and flower color underwent a definite change (see Non-Patent Document 5). When F3′5′H gene derived from petunia, gentian, butterfly pea, cineraria, eustoma, pansy and lavender were expressed in roses, delphinidin was produced at a level equal to 10% of the total amount of anthocyanidins only in the case of having expressed the F3′5′H gene derived from pansy, thereby indicating that pansy-derived F3′5′H gene functions favorably in roses (see Patent Document 4).

A genetically modified chrysanthemum that accumulated 50% or more of delphinidin and in which flower color changed to blue was obtained for the chrysanthemum varieties of Improved Reagan and Dark Splendid Reagan by transcribing pansy-derived F3′5′H gene using rose chalcone synthase promoter (see Patent Document 5). When F3′5′H gene derived from such plants as campanula, verbena, cineraria, pansy, petunia or lobelia was expressed using a promoter derived from chrysanthemum-derived flavonone 3-hydroxylase gene and a 5′-untranslated region of tobacco alcohol dehydrogenase gene, the F3′5′H genes derived from campanula, verbena, cineraria and pansy functioned comparatively well, and delphinidin was produced at a level equal to 25% of the total amount of anthocyanidins. In chrysanthemums, the campanula-derived F3′5′H gene functioned most favorably, and delphinidin was produced at a level of 75% of the total amount of anthocyanidins (see Non-Patent Document 16).

On the basis of these reported examples, although examples have been reported of producing delphinidin by expressing a heterologous (foreign) gene in plants, since it is difficult to predict which F3′5′H gene will actually be expressed in the petals of a plant of interest when F3′5′H gene derived from a certain plant is introduced into that plant of interest using a certain promoter, considerable trial-and-error and repeated experiments are required in order to produce delphinidin in petals.

In addition, all of the research results reported in the aforementioned examples were obtained from dicotyledons. There are considerable differences between dicotyledons and monocotyledons even among higher plants.

Firstly, dicotyledons and monocotyledons have various morphological differences. This indicates that monocotyledons have undergone considerable morphological evolution at their origin. Characteristics of monocotyledons consist of: having a single cotyledon, the base of the cotyledon forms a sheath that envelops the other portions of the germ, fibrovascular bundles are sporadically present in cross-sections of the stem, the stem does not become any thicker once it has finished extending (a cambium does not develop), the veins are in the form of parallel lines (parallel veins), a main root does not develop to a great extent, but rather roots are in the form of fibrous roots, the number of tepals, stamen and carpels is a multiple of 3, there is no distinction between petals and sepals (see http://www.fukuoka-edu.ac.jp/˜fukuhara/keitai/9-1.html).

Moreover, since monocotyledons and dicotyledons differ considerably in control of gene expression (see Non-Patent Document 6 and Non-Patent Document 7), difficulties are encountered when cultivating genetically modified monocotyledons. However, considerable research and development has been conducted on the monocotyledons of corn and rice, genetically modified corn has already been commercialized in numerous countries.

In this manner, although there are examples of reports describing temporary expression of heterologous (foreign) F3′5′H gene in the petals of monocotyledon flowering plants (see Patent Document 3), there are few reports describing the development of genetically modified monocotyledon flowering plants, and there are still no reports describing the development of monocotyledon flowering plants into which F3′5′H gene has been introduced, namely the development of monocotyledon flowering plants in which flower color has been altered through accumulation of delphinidin in the petals thereof as a result of introducing heterologous F3′5′H gene.

Furthermore, it is described in Non-Patent Document 8 that, when a gene relating to blue pigment synthesis was introduced into red lilies for the purpose of developing blue lilies, lilies were obtained in which the content of blue pigment accounted for one half or more of all pigment. However, the tissue or portion of the lily plants for which blue pigment was analyzed in Non-Patent Document 8 was the petiole. There are many plants in which anthocyanins are not synthesized in flowers even though they are synthesized in leaves (such as thale cress), and there are known cases in which the molecular species of anthocyanins in leaves and anthocyanins in flowers are different. For example, although anthocyanins derived from cyanidin are only contained in the leaves of Sarrecenia purpurea and Sarracenia psitticina, anthocyanins are contained in the flowers thereof that are derived from cyanidin and delphinidin (see Non-Patent Document 17). In addition, control of anthocyanin synthesis most likely also differs according to tissue. For example, although gene c imparts white flowers in morning glories, the stems are colored by anthocyanins while the seeds are black. On the other hand, although gene ca imparts white flowers, the stems are not colored and the seed are white.

Thus, the report describing that delphinidin content accounts for one half or more of all pigment in the petiole of lilies as a result of introducing a heterologous gene does not mean that a method for cultivating blue lilies was established that enables the cultivation of lilies in which delphinidin has accumulated in the petals as a result of introducing that heterologous gene.

In addition, in order to change flower color to blue, the required content of delphinidin is 50% or more, preferably 69% or more, more preferably 70% or more, even more preferably 80% or more, still more preferably 90% or more, more preferably still 95% or more, even more preferably still 99% or more, and most preferably 100%, of the total amount of anthocyanidins. Since it is frequently inadequate to simply overexpress F3′S′H gene in order to achieve this, further contrivances are necessary, including additional gene manipulation.

For example, in the case of carnations, delphinidin content becomes nearly 100% and genetically modified carnations are obtained in which flower color has been changed to blue by expressing both F3′5′H gene and petunia dihydroflavonol 4-reductase (DFR) gene in white carnations deficient in DFR gene (see Patent Document 6 and FIG. 1 of the present application). In addition, in roses, delphinidin content becomes nearly 100% and genetically modified roses are obtained in which flower color has been changed to blue by overexpressing pansy-derived F3′5′H gene and iris-derived DFR gene in addition to suppressing expression of rose DFR gene (see Patent Document 2). In addition, in chrysanthemums, delphinidin content is increased by overexpressing pansy-derived F3′5′H gene and suppressing expression of chrysanthemum endogenous F3′S′H gene (see Patent Document 5).

### PRIOR ART DOCUMENTS

### Patent Documents

Patent Document 1: International Publication No. WO 94/28140 (PCT/AU94/00265, Japanese Unexamined Patent Publication (Translation of PCT Application) No. H8-511683)
Patent Document 2: International Publication No. WO 2005/017147 (PCT/JP2004/001958)
Patent Document 3: Japanese Unexamined Patent Publication No. 2008-253250
Patent Document 4: International Publication No. WO 2004/020637 (PCT/AU03/01111)
Patent Document 5: International Publication No. WO 2009/062253 (PCT/AU2008/001694)
Patent Document 6: International Publication No. WO 96/36716 (PCT/AU96/00296)
Patent Document 7: International Publication No. WO 2007/094521 (PCT/JP2007/053342)
Patent Document 8: International Publication No. WO 2008/156214 (PCT/JP2008/061603)
Patent Document 9: Soon to be published Japanese Patent Application No. 2008-276029

### Non-Patent Documents

Non-Patent Document 1: Plant J. (2008) 54, 737-749
Non-Patent Document 2: Nature (1993) 366, 276-279
Non-Patent Document 3: FEBS Lett. (1999) 461, 241-245
Non-Patent Document 4: Biosci. Biotechnol. Biochem. (2003) 67(1), 161-165
Non-Patent Document 5: Plant Biotechnology (2006) 23, 5-11
Non-Patent Document 6: Protein, Nucleic Acid and Enzyme (1989) 34, 1873-1878
Non-Patent Document 7: Trends in Genetics (1988) 4, 13-18
Non-Patent Document 8: 2006 Annual Report No. 1-26 of the Niigata Agricultural Research Institute, (32) Project for the Development of Top Brand Agricultural Products, Cultivation of Blue Lilies
Non-Patent Document 9: Plant Cell Reports (2004) 22, 415-421
Non-Patent Document 10: Plant Cell Reports (2004) 22, 359-364
Non-Patent Document 11: Theor. App. Genet. (1999) 99, 383-390
Non-Patent Document 12: Plant Cell Physiol. (1996) 37, 49-59
Non-Patent Document 13: Clontech, Molecular Breeding (2003) 11, 287-293
Non-Patent Document 14: J. Japan Soc. Hort. Sci. (2008) 77, 94-102
Non-Patent Document 15: Plant J. (2008) 54, 949-962
Non-Patent Document 16: Abstracts of the 2010 Annual Meeting of the Japanese Society of Plant Physiologists, Poster No. P1C012(600), http://www.jspp.org/13member/2010abstract/pdf/07.pdf
Non-Patent Document 17: Hort. Science (2001) 36, 384

### SUMMARY OF THE INVENTION

### Problem to be Solved by the Invention

In plants, the anthocyanin-based pigment, delphinidin, is involved in blue color in nearly all cases. Although F3′5′H is essential for the synthesis of delphinidin, this enzyme is absent in lilies. Consequently, blue lilies do not exist. In order to cultivate blue lilies by gene introduction, F3′5′H is required to be highly expressed in lilies. On the other hand, delphinidin content does not increase in lilies synthesizing cyanidin unless synthesis of cyanidin is suppressed.

An object of the present invention is to provide a method for producing lilies that synthesize the blue pigment, delphinidin, at a high content ratio of petal cells by highly expressing F3′5′H derived from a specific species in lily petal cells together with suppressing synthesis of lily endogenous cyanidin.

### Means for Solving the Problems

As a result of conducting extensive studies to solve the aforementioned problems, the inventors of the present invention found that, when campanula-derived F3′5′H gene is introduced into the Oriental lily variety “Acapulco”, which has pink color and in which cyanidin accounts for nearly all of the anthocyanin-based pigment present, and synthesis of cyanidin is suppressed using RNAi by introducing a fragment of lily F3′H gene, 80% of all anthocyanin-based pigment in the petal cells becomes delphinidin, thereby leading to completion of the present invention.

Namely, the present invention is as indicated below.

[1] A method for producing lilies containing delphinidin in the petals thereof, comprising the following steps:

introducing into lilies a campanula-derived flavonoid 3′,5′-hydroxylase (F3′5′H) gene composed of a nucleotide sequence represented by SEQ ID NO: 1 or SEQ ID NO: 11, or a nucleotide sequence having at least 90% sequence identity with the nucleotide sequence represented by SEQ ID NO: 1 or SEQ ID NO: 11 that also encodes a polypeptide having F3′5′H activity, introducing a fragment of lily-derived flavonoid 3′-hydroxylase (F3′H) gene composed of a nucleotide sequence represented by SEQ ID NO: 3 or SEQ ID NO: 16, or a nucleotide sequence having at least 90% sequence identity with the nucleotide sequence represented by SEQ ID NO: 3 or SEQ ID NO: 16 that also encodes a polypeptide having F3′H activity, and synthesizing delphinidin by the action of the introduced F3′5′H gene while suppressing expression of endogenous F3′H gene that acts on cyanidin synthesis in lily petals.

[2] The method described in [1] above, wherein the suppression of endogenous F3′H expression is carried out in the RNAi method.

[3] The method described in [1] or [2] above, wherein the campanula-derived F3′5′H gene is composed of a nucleotide sequence having at least 95% sequence identity with the nucleotide sequence represented by SEQ ID NO: 1 or SEQ ID NO: 11 that also encodes a polypeptide having F3′5′H activity.

[4] The method described in [1] or [2] above, wherein the campanula-derived F3′5′H gene is composed of a nucleotide sequence represented by SEQ ID NO: 1 or SEQ ID NO: 11.

[5] The method described in any of [1] to [4] above, wherein the fragment of lily-derived F3′H gene is composed of a nucleotide sequence having at least 95% sequence identity with the nucleotide sequence represented by SEQ ID NO: 3 or SEQ ID NO: 16 that also encodes a polypeptide having F3′H activity.

[6] The method described in any of [1] to [4] above, wherein the fragment of lily-derived F3′H gene is composed of a nucleotide sequence represented by SEQ ID NO: 3 or SEQ ID NO: 16.

[7] The method described in any of [1] to [6] above, further comprising: introducing a fragment of lily-derived dihydroflavonol 4-reductase (DFR) gene composed of a nucleoide sequence represented by SEQ ID NO: 13 or a base sequence having at least 90% sequence identity with the nucleotide sequence represented by SEQ ID NO: 13 that also encodes a polypeptide having DFR activity, suppressing expression of endogenous DFR that acts on cyanidin synthesis in lily petals, and simultaneously introducing an iris-derived foreign DFR gene.

[8] The method described in [7] above, wherein the suppression of expression of endogenous DFR is carried out in the RNAi method.

[9] The method described in any of [1] to [8] above, further comprising: using a lily DFR promoter sequence represented by SEQ ID NO: 24.

[10] A lily, tissue thereof, portion thereof, vegetatively propagated plant thereof or progeny thereof, which contains delphinidin in the petals thereof produced according to the method described in any of [1] to [9] above.

[11] The lily, tissue thereof, portion thereof, vegetatively propagated plant thereof or progeny thereof described in [10] above, wherein the tissue or portion thereof is a cut flower.

### EFFECTS OF THE INVENTION

According to the method for producing lilies containing delphinidin in the petals thereof according to the present invention, a blue pigment present in lily petal cells in the form of delphinidin can be made to accumulate at a high content ratio, thereby making it possible to produce previously non-existent lilies that produce blue flowers.

### BRIEF DESCRIPTION OF THE DRAWINGS

FIG. 1 indicates the biosynthesis pathway of anthocyanins.

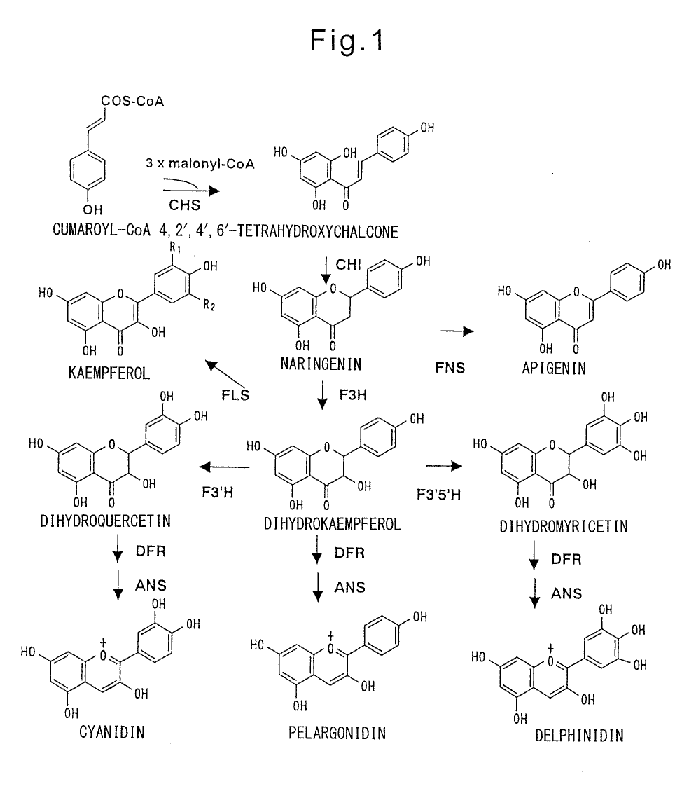

FIG. 2 indicates a map of a binary vector Tr3.

FIG. 3 indicates maps of binary vectors Tr7 to Tr11.

### EMBODIMENTS OF THE INVENTION

The following provides a detailed explanation of the present invention.

The present invention relates to a method for producing lilies containing delphinidin in the petals thereof, comprising the following steps:

introducing into lilies a campanula-derived flavonoid 3′,5′-hydroxylase (F3′5′H) gene composed of a nucleotide sequence represented by SEQ ID NO: 1 or SEQ ID NO: 11, or a nucleotide sequence having at least 90% sequence identity with the nucleotide sequence represented by SEQ ID NO: 1 or SEQ ID NO: 11 that also encodes a polypeptide having F3′5′H activity, introducing a fragment of lily-derived flavonoid 3′-hydroxylase (F3′H) gene composed of a nucleotide sequence represented by SEQ ID NO: 3 or SEQ ID NO: 16, or a nucleotide sequence having at least 90% sequence identity with the nucleotide sequence represented by SEQ ID NO: 3 or SEQ ID NO: 16 that also encodes a polypeptide having F3′H activity, and synthesizing delphinidin by the action of the introduced F3′5′H gene while suppressing expression of endogenous F3′H gene that acts on cyanidin synthesis in lily petals.

Examples of the aforementioned nucleotide sequence having sequence identity of at least 90% with a nucleotide sequence represented by SEQ ID NO: 1 or SEQ ID NO: 11 that also encodes a polypeptide having flavonoid 3′5′-hydroxylase (F3′5′H) activity include DNA having sequence identity of least 91%, 92%, 93%, 94%, 95%, 96%, 97%, 98% and 99% or more with DNA composed of a nucleotide sequence represented by SEQ ID NO: 1 when calculated using analysis software such as BLAST or FASTA.

Similarly, examples of the aforementioned nucleotide sequence having at least 90% sequence identity with a nucleotide sequence represented by SEQ ID NO: 3 or SEQ ID NO: 16 that also encodes a polypeptide having flavonoid 3′-hydroxylase (F3′H) activity include DNA having sequence identity of at least 91%, 92%, 93%, 94%, 95%, 96%, 97%, 97%, 98% and 99% or more with DNA composed of a nucleotide sequence represented by SEQ ID NO: 3 when calculated using analysis software such as BLAST or FASTA.

The nucleotide sequence having at least 90% sequence identity with a nucleotide sequence represented by SEQ ID NO: 1 or SEQ ID NO: 11 that has flavonoid 3′,5′-hydroxylase (F3′5′H) activity can also be a nucleotide sequence in which one or more nucleotides have been deleted, substituted or added in a nucleotide sequence represented by SEQ ID NO: 1 or SEQ ID NO: 11 that also encodes a polypeptide having flavonoid 3′,5′-hydroxylase (F3′5′H) activity. Here, “one or more” in DNA or a nucleotide sequence refers to 1 to 20, preferably 1 to 10, more preferably 1 to 5, even more preferably 1 to 4, and still more preferably 1 to 3 of the bases in the nucleotide sequence represented by SEQ ID NO: 1 being deleted, substituted or added provided it is within the range of having the aforementioned enzyme activity. In addition, the terms “deletion”, “substitution” and “addition” refer to the formation of a nucleotide sequence that encodes a protein having properties similar to a protein (or polypeptide) (SEQ ID NO: 2) encoded by the nucleotide sequence represented by SEQ ID NO: 1.

Similarly, the nucleotide sequence having at least 90% sequence identity with a nucleotide sequence represented by SEQ ID NO: 3 or SEQ ID NO: 16 that has flavonoid 3′-hydroxylase (F3′H) activity can also be a nucleotide sequence in which one or more nucleotides have been deleted, substituted or added in a nucleotide sequence represented by SEQ ID NO: 3 or SEQ ID NO: 16 that also encodes a polypeptide having flavonoid 3′-hydroxylase (F3′H) activity. Here, “one or more” in DNA or a nucleotide sequence refers to 1 to 20, preferably 1 to 10, more preferably 1 to 5, even more preferably 1 to 4, and still more preferably 1 to 3 of the nucleotides in the nucleotides sequence represented by SEQ ID NO: 3 or SEQ ID NO: 16 being deleted, substituted or added provided it is within the range of having the aforementioned enzyme activity. In addition, the terms “deletion”, “substitution” and “addition” refer to the formation of a nucleotide sequence that encodes a protein having properties similar to a protein (or polypeptide) (SEQ ID NO: 4 or SEQ ID NO: 17) encoded by the nucleotide sequence represented by SEQ ID NO: 1 or SEQ ID NO: 16.

The nucleotide sequence having sequence identity of at least 90% with a nucleotide sequence represented by SEQ ID NO: 1 or SEQ ID NO: 11 that also encodes a polypeptide having flavonoid 3′,5′-hydroxylase (F3′5′H) activity can be a nucleotide sequence capable of hybridizing under stringent conditions with a complementary strand of a nucleotide sequence represented by SEQ ID NO: 1 or SEQ ID NO: 11 that also encodes a polypeptide having flavonoid 3′5′-hydroxylase (F3′5′H) activity.

Similarly, the nucleotide sequence having sequence identity of at least 90% with a nucleotide sequence represented by SEQ ID NO: 3 or SEQ ID NO: 16 that also encodes a polypeptide having flavonoid 3′-hydroxylase (F3′H) activity can be a nucleotide sequence capable of hybridizing under stringent conditions with a complementary strand of a nucleotide sequence represented by SEQ ID NO: 3 that also encodes a polypeptide having flavonoid 3′-hydroxylase (F3′H) activity.

Here, although the temperature and salt concentration during hybridization, and preferably during washing as well, can be suitably set corresponding to the DNA that is hybridized to a complementary strand of DNA composed of SEQ ID NO: 1 or 11 or SEQ ID NO: 3 or 16, “stringent conditions” with respect to a nucleotide or DNA (sequence) refers to conditions described in, for example, “Molecular Cloning: A Laboratory Manual (2nd Edition)” edited by Sambrook et al. (Cold Spring Harbor Laboratory Press (1989). More specifically, this refers to a step consisting of (i) incubating overnight at 42° C. with a probe in a solution containing 6×SSC (composition of 1×SSC: 0.15 M NaCl, 0.015 M sodium citrate, pH 7.0), 0.5% SDS, 5×Denhardt's solution, 100 μg/mL denatured fragmented salmon sperm DNA and 50% formaldehyde, and (ii) removing non-specifically hybridized probe by washing, wherein from the viewpoint of further enhancing accuracy, washing under conditions of lower ionic strength such as 2×SSC and more stringently at 0.1×SSC, and/or washing at a high temperature, such as 40° C. lower than the Tm value of the nucleic acid used, more stringently at 30° C. lower, even more stringently at 25° C. lower and still more stringently at 10° C. lower, and more specifically, although varying according to the Tm value of nucleic acid used, washing under conditions of 25° C. or higher, more stringently at 37° C. or higher, even more stringently at 42° C. or higher, still more stringently at 50° C. or higher, and even more stringently still at 60° C. or higher. Tm is determined from, for example, the formula Tm=81.5+16.6(log [Na⁺])+0.41(% G+C)−(600/N) (wherein, N represents oligonucleotide length, and % G+C represents the contents of guanine and cytosine residues in the oligonucleotide). Hybridization conditions may be set by, for example, referring to the aforementioned publication. Furthermore, the procedure described in the present description can be suitably carried out by referring to the aforementioned publication.

Suppression of expression of the endogenous flavonoid 3′-hydroxylase (F3′H) acting on cyanidin synthesis in lily petals by introducing a fragment of lily-derived F3′H gene composed of a sequence represented by SEQ ID NO: 3 or SEQ ID NO: 16 or a sequence having at least 90% sequence identity with a nucleotide sequence represented by SEQ ID NO: 3 or SEQ ID NO: 16 that also encodes a polypeptide having F3′H activity may be carried out by antisense suppression, sense suppression (co-suppression) or RNAi and the like, the RNAi method is preferable.

The present invention also relates to the aforementioned method further comprising inserting a fragment of lily-derived dihydroflavonol 4-reductase (DFR) gene composed of a nucleotide sequence represented by SEQ ID NO: 13 or a nucleotide sequence having at least 90% sequence identity with the nucleotide sequence represented by SEQ ID NO: 13 that also encodes a polypeptide having DFR activity, suppressing expression of endogenous DFR that acts on cyanidin synthesis in lily petals, and simultaneously introducing an iris-derived foreign DFR gene. The suppression of expression of endogenous DFR is preferably carried out in the RNAi method.

The present invention also relates to the aforementioned method further comprising the step of using a lily DFR promoter represented by SEQ ID NO: 24.

Furthermore, the definitions of the deletion, substitution or addition of one or more nucleotides in a nucleotide sequence with respect to SEQ ID NO: 1, 11, 3 and 16, sequence identify and hybridization are similarly also applied to SEQ ID NO: 13 and 24, and instead of the nucleotide sequences represented by SEQ ID NO: 13 and 24, for example, a nucleotide sequence having sequence identity of at least 90%, 95% or 99% therewith is also within the scope of the present invention.

A lily containing delphinidin in the petals thereof produced according to the method for producing lilies containing delphinidin in the petals thereof according to the present invention, tissue thereof, a portion thereof, a vegetatively propagated plant thereof or progeny thereof, is within the scope of the present invention. The tissue or portion thereof can preferably be a cut flower.

### EXAMPLES

Although the following provides a more detailed explanation of the present invention through examples thereof, the scope of the present invention is not intended to be limited by these examples.

### Reference Example 1

### Expression of Periwinkle F3′5′H Gene in Lilies

Plasmid pNAVFH (see Non-Patent Document 9, binary vector for expressing periwinkle F3′S′H gene in plants) was introduced into Agrobacterium strain EHA101. The lily variety, Acapulco (pink flowers), was transformed using this genetically modified Agrobacterium. Although lily transformation was carried out with the method described in Non-Patent Document 10, for example, the method used is not limited thereto.

Two to three years are required after acquiring a plant tissue culture of the transformed lilies in order to investigate whether the introduced F3′5′H gene functions in lilies and induces accumulation of delphinidin. Analyzing anthocyanins present in cultured petioles by focusing on observation of anthocyanin coloring in the petioles at least makes it possible to rapidly determine whether the introduced gene has not functioned or has hardly functioned at all. Plant tissue cultures of the transformed lilies of the resulting 19 cutivars were acquired. Approximately 0.5 g of these petioles in which anthocyanin coloring was observed were recovered and subjected to anthocyanidin analysis using the method described in Patent Document 2. As a result, there were no cultivars observed in which delphinidin was detected.

### Reference Example 2

### Expression of Cineraria F3′5′H Gene in Lilies

An approximately 1.7 kb DNA fragment obtained by digesting plasmid pSPB2774 containing cineraria-derived F3′5′H cDNA Ci5a18 (see Patent Document 9) with SmaI and XhoI, a DNA fragment containing cauliflower mosaic virus 35S promoter (to also be referred to as “35S promoter”) obtained by digesting plasmid pBI1221 with HindIII and BamHI, and a DNA fragment containing the replicon of a DNA fragment obtained by digesting plasmid pSPB176 (see Patent Document 7) with HindIII and SalI, were ligated to obtain plasmid pSPB3472. A DNA fragment obtained by digesting this plasmid with AscI and PacI (containing a sequence in which 35S promoter, Ci5a18 and nopaline synthase terminator are ligated) was ligated with a DNA fragment obtained by digesting #493 with AscI and PacI to obtain plasmid pSPB3376. This was then introduced into lilies using Agrobacterium as described in Reference Example 1 to acquire plant tissue cultures of 65 cultivars of transformed lilies. Although leafstalks in which anthocyanins had accumulated were acquired from 23 of these cultivars, there were no cultivars obtained in which delphinidin had accumulated.

### Reference Example 3

### Expression of Gentian F3′5′H Gene in Lilies

An approximately 1.7 kb DNA fragment obtained by digesting plasmid pGHF48 containing gentian F3′5′H cDNA (see Patent Document 4) was ligated with plasmid pSPB176 digested with BamHI and SalI to obtain plasmid pSPB3329. The promoter portion was removed by digesting this with HindIII and BamHI. Plasmid pSPB3473 was then obtained by introducing a DNA fragment obtained by digesting pBI1221 with HindIII and BamHI (containing 35S promoter) therein. A DNA fragment obtained by digesting this plasmid with AscI and PacI (containing a sequence in which 35S promoter, gentian F3′5′H cDNA and nopaline synthase terminator are ligated) was ligated with a DNA fragment obtained by digesting pSPB493 with AscI and PacI to obtain plasmid pSPB3378. This was then introduced into lilies using Agrobacterium as described in Reference Example 1 to acquire plant tissue cultures of 65 strains of transformed lilies. Petioles in which anthocyanins had accumulated were acquired from 26 of these cultivars. The delphinidin accumulation ratio (ratio of the amount of delphinidin accumulated to the total amount of accumulated anthocyanidins) only reached a maximum of 1%.

### Reference Example 4

### Expression of Lavender F3′5′H Gene in Lilies

An approximately 1.7 kb DNA fragment obtained by digesting plasmid pLBG8 containing lavender F3′5′H cDNA (see Patent Document 4) with BamHI and XhoI was ligated with plasmid pSPB176 digested with BamHI and SalI to obtain plasmid pSPB2772. A DNA fragment containing a sequence in which lavender F3′5′H cDNA and nopaline synthase terminator were ligated and obtained by digesting this plasmid with BamHI and PacI was recovered. This DNA fragment was ligated with a fragment containing the replicon of a DNA fragment obtained by digesting plasmid pSPB3472 (3377) with BamHI and PacI to obtain plasmid pSPB3471. A DNA fragment obtained by digesting this plasmid with AscI and PacI (containing a sequence in which 35S promoter, gentian F3′5′H cDNA and nopaline synthase terminator are ligated) was ligated with a DNA fragment obtained by digesting pSPB493 with AscI and PacI to obtain plasmid pSPB3376. This was then introduced into lilies using Agrobacterium as described in Reference Example 1 to acquire plant tissue cultures of 23 sultivars of transformed lilies. Petioles in which anthocyanins had accumulated were acquired from 20 of these cultivars. The delphinidin accumulation ratio (ratio of the amount of delphinidin accumulated to the total amount of accumulated anthocyanidins) only reached a maximum of 1%.

### Reference Example 5

### Expression of Butterfly Pea F3′5′H Gene in Lilies

Introduction of butterfly pea F3′5′H gene (DNA Database Accession No. AB234897) into verbena has been reported to function better and result in a higher delphinidin content and a more distinct change in flower color than in the case of introducing the F3′5′H gene of verbena per se (see Non-Patent Document 5). Plasmid pSPB748 (see Non-Patent Document 5) was digested with EcoRI, subjected to blunting and digested with BamHI followed by recovering a DNA fragment containing butterfly pea-derived F3′5′H cDNA. On the other hand, a DNA fragment was obtained by digesting pSPB176 with SalI, blunting and digesting with BamHI. These two DNA fragments were ligated to obtain plasmid pSPB3166. A DNA fragment containing F3′5′H cDNA obtained by digesting pSPB3166 with AscI and PacI was inserted into a DNA fragment obtained by digesting binary vector pSBP3166, which contains a gene imparting hygromycin resistance (see Non-Patent Document 11), with AscI and PacI. The resulting binary vector was designated as pSBP3169. In plants transformed with this binary vector, butterfly pea F3′5′H gene is transcribed under the control of cauliflower mosaic virus 35S promoter to which an enhancer has been added (see Non-Patent Document 12).

This was then introduced into lilies using Agrobacterium as described in Reference Example 1, and plant tissue cultures of 23 cultivars of transformed lilies were acquired in anticipation of production of delphinidin following introduction of butterfly pea F3′5′H gene into lilies. Although petioles in which anthocyanins had accumulated were acquired from 6 of these cultivars, there were no cultivars obtained in which delphinidin had accumulated.

### Reference Example 6

### Expression of Pansy F3′5′H Gene in Lilies

In the case of having introduced F3′5′H genes derived from several plants into roses, only pansy-derived F3′5′H gene (DNA Database Accession No. AB332097) resulted in favorable accumulation of delphinidin and caused a distinct change in flower color (see Patent Document 4). In addition, this gene also causes delphinidin to accumulate in chrysanthemum petals and causes flower color to change to blue (see Patent Document 5). On the basis of these facts, pansy-derived F3′5′H gene was presumed to be suitable for producing delphinidin in different plants.

A DNA fragment obtained by digesting plasmid pSPB575 containing pansy F3′5′H cDNA (see Patent Document 8) with AscI and PacI and a DNA fragment obtained by digesting plasmid pSPB3166 with AscI and PacI were ligated to obtain binary vector pSBP3138. In plants transformed with this binary vector, pansy F3′5′H gene is transcribed under the control of cauliflower mosaic virus 35S promoter to which an enhancer has been added.

This was then introduced into lilies using Agrobacterium as described in Reference Example 1, and plant tissue cultures of 25 cultivars of transformed lilies were acquired in anticipation of production of delphinidin following introduction of pansy F3′5′H gene into lilies. Although petioles in which anthocyanins had accumulated were acquired from 12 of these cultivars, there were no cultivars obtained in which delphinidin had accumulated.

### Example 1

### (1) Isolation of F3′5′H Gene from Campanula Genomic DNA

Chromosomal DNA was extracted from leaves of Campanula (Campanula medium) using DNeasy (Qiagen Corp.). Nucleotides CFH1L: 5′-CATGTCTATAGACATATCCACCCTCTTCTATGAAC-3′ (SEQ ID NO: 5, underlines indicate start codon) and cCFH1L: 5′-GCCTAGACAGTGTAAGAACTTGGAGGCAATCTTGG-3′ (SEQ ID NO: 6) were synthesized based on the base sequence of campanula (Campanula medium) F3′S′H cDNA (DNA Database Accession No. D14590). PCR was carried out using 100 ng of chromosomal DNA as template and using the aforementioned nucleotides as primers. Takara LA PCR (Takara Bio Inc.) was used to carry out PCR under the conditions recommended by the manufacturer. The resulting base sequence is represented by SEQ ID NO: 1. Amino acid SEQ ID NO: 2 encoded by this base sequence is the same as that registered for DNA Database Accession No. D14590.

### (2) Construction of Binary Vector Containing F3′S′H Gene from Campanula Genomic DNA

Plasmid pBI221 (see Non-Patent Document 13) was digested with restriction enzymes SmaI and SacI followed by blunting using a blunting kit (Takara Bio Inc.) and subjecting to ligation. The resulting plasmid was digested with restriction enzyme BamHI and blunted. This DNA fragment was then reacted with DNA polymerase in the presence of deoxythymidine triphosphate to add a single deoxythymidine base to the 3′-end of this DNA fragment. This DNA fragment was then ligated with PCR fragments derived from the aforementioned campanula chromosomal DNA. Among the resulting plasmids, the plasmid in which the start codon of campanula F3′5′H gene was inserted in a direction close to a 35S promoter sequence was designated as p35S-CFH-1. This plasmid was digested with PvuII and separated by agarose gel electrophoresis to recover a DNA fragment containing campanula F3′5′H gene.

Plasmid pNAVFH (see Non-Patent Document 9, binary vector for expressing periwinkle F3′5′H gene in plants) was digested with restriction enzymes HindIII and XbaI, blunted and dephosphorylated. This was then separated by agarose gel electrophoresis followed by recovery of the DNA fragment having the largest molecular weight. This DNA fragment was ligated with the aforementioned DNA fragment containing campanula F3′5′H gene, and the resulting binary vector was designated as pNARI-CFH.

### (3) Isolation of Lily F3′H Gene Fragment (Construction of pCR2.1-AP24-1)

Oligonucleotides F3H2: 5′-YTSGCYGGWGTWTTYAACRTHGG-3′, equivalent to LAGVFNIG in the sequence of thale cress (SEQ ID NO: 7), and cF3H4: 5′-GGRTCNCGRGCWATGGCCCA-3′, equivalent to NIWAIARDP in the sequence of thale cress (SEQ ID NO: 8), were synthesized based on the previously reported F3′H amino acid sequences of thale cress (DNA Database Accession No. AF271651), aster (AF313488), soybean (AF499731), perilla (AB045593), rice (AC021892) and torenia (AB057672). Here, Y represents a mixture of C and T, W represents a mixture of A and T, S represents a mixture of C and G, R represents a mixture of A and G, H represents a mixture of A, T and C, and N represents a mixture of G, A, T and C. RNA was extracted from the petals of the lily variety, Acapulco (Yamaki Noen Co., Ltd.), using RNeasy (Qiagen Corp.). RT-PCR was carried out using this RNA as template and using F3H2 and oligo(dT) as primers. RT-PCR was carried out using an RT-PCR kit (Invitrogen Corp.). PCR was carried out as previously described using the amplified DNA fragment as template and using F3H2 and cF3H4 as primers. The resulting DNA fragment was designated as SEQ ID NO: 3. A DNA fragment having this sequence was then ligated to plasmid pCR2.1 (Invitrogen Corp.). The resulting plasmid was designated as pCR2.1-AP24-1.

### (4) Construction of Plasmid pNARI-Tr3

Plasmid pCR2.1-AP24-1 was cleaved with SpeI and XbaI, blunted and separated by agarose gel electrophoresis. A DNA fragment having a length of 665 bp was recovered from the resulting DNA fragments. This DNA fragment was ligated with a DNA fragment obtained by cleaving plasmid pBI221 with restriction enzymes ScaI and MluI, blunting and subjecting to dephosphorylation treatment. The resulting plasmid was designated as p35S-GUSL-AP24.

Plasmid pCR2.1-AP24-1 was cleaved with SpeI and NotI, blunted and separated by agarose gel electrophoresis. A 647 bp DNA fragment was recovered from the resulting DNA fragments. This DNA fragment was ligated with a DNA fragment obtained by digesting plasmid p35S-GUSL-AP24 with SmaI and subjecting to dephosphorylation treatment, and the resulting plasmid was designated as plasmid p35S-F3′HTr2.

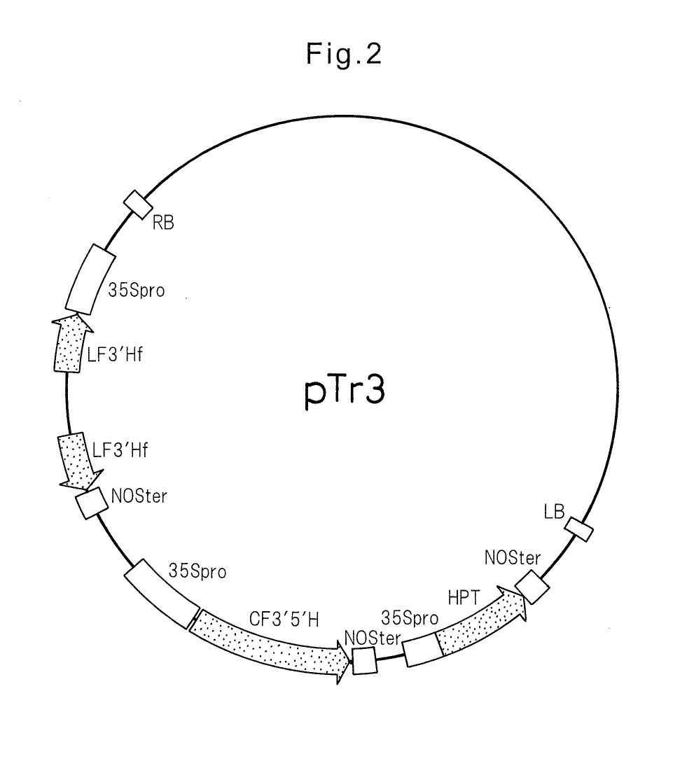

Plasmid p35S-F3′HTr2 was cleaved with PvuII and separated by agarose gel electrophoresis followed by recovery of an approximately 3.3 kb DNA fragment. On the other hand, plasmid pNARI-CFH was cleaved with ClaI and NheI, blunted, dephosphorylated and separated by agarose gel electrophoresis followed by recovery of a DNA fragment containing the replicon. The recovered DNA fragment was ligated with the aforementioned approximately 3.3 kb DNA fragment. The resulting plasmid or binary vector was designated as pNARI-Tr3 (which may also be simply referred to as “Tr3”). As shown in FIG. 2, this binary vector has a hygromycin phosphotransferase gene on T-DNA as a selection marker, and it was intended to suppress expression of lily F3′H gene by constitutively expressing campanula F3′5′H gene and transcribing double-stranded RNA of lily F3′H gene.

### (5) Lily Transformation

Binary vectors pNARI-CFH and pNARI-Tr3 were each introduced into Agrobacterium strain EHA101. Lily variety Acapulco (pink flowers) was transformed using these genetically modified Agrobacterium as described in Reference Example 1. In the case of using Agrobacterium containing pNARI-CFH, plant tissue cultures of 31 strains of lilies were acquired. Analysis of anthocyanidins in the leafstalks where anthocyanins had accumulated revealed that delphinidin had accumulated at a level of 5% or more of the total amount of anthocyanidins in 4 strains. The maximum delphinidin content was 18%.

On the other hand, in the case of using Agrobacterium containing pNART-Tr3, plant tissue cultures of 40 strains of lilies were acquired. Analysis of anthocyanidins in the leafstalks where anthocyanins had accumulated revealed that delphinidin had accumulated at a level of 5% or more of the total amount of anthocyanidins in 13 cultivars as shown in the following Table 1. When several of these genetically modified lilies were allowed to bloom in a greenhouse enabling the cultivation of genetically modified plants, the content of delphinidin in the petals reached a maximum of 80%, and in contrast to the original color of this variety having a color chart number of N74D as classified by the British Royal Horticultural Society, the color of the genetically modified lilies become bluer in comparison with the original color, and demonstrated a color chart number of 81C.

TABLE 1

| | Petiole Anthocyanidin | Petal Anthocyanidin |
|---|---|---|
| | Analysis | Analysis |
| | (delphinidin/cyanidin/ | (delphinidin/cyanidin/ |
| Individual No. | pelargonidin: %) | pelargonidin: %) |
| AP Tr3 No. 1 | 4/96/0 | 0/96/4 |
| AP Tr3 No. 2 | 3/97/0 | 1/84/15 |
| AP Tr3 No. 3 | 26/63/11 | Did not bloom |
| AP Tr3 No. 4 | 0/100/0 | Did not bloom |
| AP Tr3 No. 5 | 53/39/8 | 80/13/7 (090323) |
| AP Tr3 No. 6 | 7/91/2 | Did not bloom |
| AP Tr3 No. 7 | 0/100/0 | Did not bloom |
| AP Tr3 No. 8 | 0/100/0 | Did not bloom |
| AP Tr3 No. 9 | 0/100/0 | Did not bloom |
| AP Tr3 No. 10 | 0/100/0 | 0/99/1 (091020) |
| AP Tr3 No. 11 | 0/100/0 | 7/78/15 (091020) |
| AP Tr3 No. 12 | 68/25/7 | 65/20/15 (080516) |
| AP Tr3 No. 13 | 0/100/0 | Did not bloom |
| AP Tr3 No. 14 | 50/40/10 | 12/53/32 (080516) |
| AP Tr3 No. 15 | 30/68/2 | Did not bloom |
| AP Tr3 No. 16 | 3/97/0 | 3/94/3 (091020) |
| AP Tr3 No. 17 | 20/77/3 | Did not bloom |
| AP Tr3 No. 18 | 50/43/7 | 21/79/0 |
| AP Tr3 No. 19 | 0/100/0 | Did not bloom |
| AP Tr3 No. 20 | 0/100/0 | Did not bloom |
| AP Tr3 No. 21 | 45/53/2 | Dead |
| AP Tr3 No. 22 | 25/70/5 | 0/82/18 |
| AP Tr3 No. 23 | 1/99/0 | 0/84/16 |
| AP Tr3 No. 24 | 5/93/2 | Did not bloom |
| AP Tr3 No. 25 | 9/89/2 | Did not bloom |
| AP Tr3 No. 26 | 30/67/3 | Dead |
| AP Tr3 No. 27 | 4/95/1 | 1/85/14 |
| AP Tr3 No. 28 | 2/98/0 | Did not bloom |
| AP Tr3 No. 29 | 3/97/0 | Did not bloom |
| AP Tr3 No. 30 | 30/69/1 | Did not bloom |
| AP Tr3 No. 31 | 4/94/2 | Did not bloom |
| AP Tr3 No. 32 | 2/98/0 | Did not bloom |
| AP Tr3 No. 33 | 10/90/0 | 53/39/8 |
| AP Tr3 No. 34 | 2/98/0 | Did not bloom |
| AP Tr3 No. 35 | 0/100/0 | Did not bloom |
| AP Tr3 No. 36 | 0/100/0 | Did not bloom |
| AP Tr3 No. 37 | 16/84/0 | Did not bloom |
| AP Tr3 No. 38 | 0/100/0 | Did not bloom |
| AP Tr3 No. 39 | 0/100/0 | Did not bloom |
| AP Tr3 No. 40 | 0/100/0 | Did not bloom |

In addition, pelargonidin was also detected in the genetically modified plants that is not observed in the host. This is presumed to be the effect of suppression of F3′H gene.

Similarly, Tiara (Yamaki Noen Co., Ltd.) (to also be referred to as “TI”), lily variety R094-19 (Niigata Agricultural Research Institute) (to also be referred to as “RO”) and MP8 (provided by Mr. Miwa Fujii) were also transformed using Agrobacterium containing pNARI-Tr3, and 38, 82 and 12 genetically modified cultivars, respectively, were obtained. Analysis of anthocyanidins in the petioles where anthocyanins had accumulated in these cultivars revealed that the average content of delphinidin was 24%, 8% and 11%, respectively, and the maximum value of delphinidin content was 68%, 53% and 35%, respectively.

Analysis values and flower color for those TI (Tiara) and R0 (R094-19) that bloomed are shown in the following Table 2. The maximum content of delphinidin in petals of the genetically modified tiara that bloomed was 74% (individual no. TI Tr3 No. 42). The maximum content of delphinidin in petals of genetically modified R094-19 that bloomed was 15% (individual no. RO Tr3 No. 9).

TABLE 2

| Individual No. | Delphinidin Content (%) | Color Chart No. |
|---|---|---|
| TI Tr3 No. 4 | 57 | 75D |
| TI Tr3 No. 24 | 47 | 76C |
| TI Tr3 No. 42 | 74 | 75D |
| TI Tr3 No. 43 | 0 | 84C |
| TI Tr3 No. 59 | 23 | 76D |
| TI Non-transformed | 0 | 75C |
| RO Tr3 No. 1 | 0 | 75B |
| RO Tr3 No. 9 | 15 | 75C |
| RO Tr3 No. 12 | 0 | NN155C |
| RO Tr3 No. 13 | 0 | NN155C |
| RO Tr3 No. 18 | 0 | NN155C |
| RO Non-transformed | 0 | 75B |

Moreover, anthocyanins present in host Acapulco variety and a genetically modified lily (also referred to as “Acapulco Tr3-5”) were analyzed by LC-FTICR-MS (see Non-Patent Document 14 and Non-Patent Document 15). The use of this technique makes it possible to precisely measure the mass spectra of anthocyanins and obtain MS/MS spectra by tandem mass spectrometry. In the host Acapulco variety, peaks corresponding to cyanidin, having detected m/z of 595.166612 (MS/MS m/z: 287.1 (10%), 432.8 (0.9%), 449.1 (14.7%) (figures in parentheses indicate relative values of peak intensity), bound with 1 equivalent of rhamnose and glucose comprised the major anthocyanins. On the other hand, in the case of Acapulco Tr3-5, two peaks corresponding to delphinidin, having detected m/z of 611.160529 (MS/MS mz: 303.1 (100%), 449.2 (1.1%), 465.1 (10.8%)) and 611.160902 (MS/MS m/z: 303.1 (100%), 449.1 (0.7%), 465.1 (12.3%)), bound with 1 equivalent of rhamnose and glucose were observed that are not observed in the host Acapulco variety. Moreover, an increase was observed in a peak corresponding to pelargonidin, which is only observed at extremely low levels in the host Acapulco variety, bound with one equivalent of rhamnose and glucose (detected m/z: 579.171847 (MS/MS m/z: 271.1 (100%), 417.1 (0.8%), 433.1 (18.4%)).

### Example 2

### Expression of Campanula F3′5′H cDNA in Lilies

(1) Isolation of Campanula F3′5′H cDNA

Two types of primers consisting of CamF1: 5′-GTGAAGCCACCATGTCTATAG-3′ (SEQ ID NO: 9) and CamR1: 5V-GCATTTGCCTAGACAGTGTAAG-3′ (SEQ ID NO: 10) were synthesized based on the translation sequence of F3′5′H cDNA of campanula (Campanula medium) registered in the GenBank DNA database (DNA Database Accession No. D14590). RNA was extracted from the petals of commercially available campanula buds using the RNeasy Mini Plant Kit (Qiagen Corp.), and 1st strand DNA was synthesized using an RT-PCR kit. PCR was then carried out using the primers while using this 1st strand DNA as template. The resulting DNA fragments were cloned in pCR-TOPO II. Among these clones, the base sequence of cDNA of clone #4 (designated as pSPB2561) was determined to be SEQ ID NO: 11. In contrast to the F3′5′H registered for DNA Database Accession No. D14590 being composed of 523 residues (SEQ ID NO: 2), the F3′5′H encoded by the cDNA of clone #4 was composed of 521 residues (SEQ ID NO: 12), and demonstrated identity of 95% with the F3′5′H registered for D14590.

Campanula genomic F3′5′H gene is contained in the binary vector introduced into lilies in Example 1. This sequence (SEQ ID NO: 1) has an intron. Binary vectors in which this genomic F3′5′H gene was replaced with a sequence derived from the aforementioned clone #4 (not containing an intron) were constructed. The binary vector not containing F3′5′H double-stranded DNA was designated as pNARI-cCFH (to also be referred to as “cC”), while the binary vector containing F3′H double-stranded DNA was designated as pNARI-cTr3 (to also be referred to as “cTr3”).

As shown in the following Table 3, in the case of using Agrobacterium containing pNARI-cCFH, plant tissue cultures of 42 cultivars of genetically modified lilies were acquired. Petioles in which anthocyanins had accumulated were acquired. Analysis of anthocyanidins in the petioles where anthocyanins had accumulated revealed that delphinidin had accumulated at a level of 5% or more of the total amount of anthocyanidins in 16 cultivars. The maximum delphinidin content was 36%.

TABLE 3

| | Petiole Anthocyanidin Analysis |
|---|---|
| Individual No. | (delphinidin/cyanidin/pelargonidin: %) |
| AP cC No. 1 | 1/99/0 |
| AP cC No. 2 | 13/87/0 |
| AP cC No. 3 | 1/99/0 |
| AP cC No. 4 | 1/99/0 |
| AP cC No. 5 | 7/93/0 |
| AP cC No. 6 | 1/99/0 |
| AP cC No. 7 | 4/96/0 |
| AP cC No. 8 | 1/99/0 |
| AP cC No. 9 | 5/95/0 |
| AP cC No. 10 | 1/99/0 |
| AP cC No. 11 | 0/100/0 |
| AP cC No. 12 | 0/100/0 |
| AP cC No. 13 | 1/99/0 |
| AP cC No. 14 | 1/99/0 |
| AP cC No. 15 | 1/99/0 |
| AP cC No. 16 | 1/99/0 |
| AP cC No. 17 | 1/99/0 |
| AP cC No. 18 | 8/92/0 |
| AP cC No. 19 | 0/100/0 |
| AP cC No. 20 | 26/74/0 |
| AP cC No. 21 | 0/100/0 |
| AP cC No. 22 | 13/87/0 |
| AP cC No. 23 | 6/94/0 |
| AP cC No. 24 | 0/100/0 |
| AP cC No. 25 | 0/100/0 |
| AP cC No. 26 | 10/90/0 |
| AP cC No. 27 | 1/99/0 |
| AP cC No. 28 | 10/90/0 |
| AP cC No. 29 | 16/84/0 |
| AP cC No. 30 | 12/88/0 |
| AP cC No. 31 | 12/88/0 |
| AP cC No. 32 | 3/97/0 |
| AP cC No. 33 | 3/97/0 |
| AP cC No. 34 | 0/100/0 |
| AP cC No. 35 | 0/100/0 |
| AP cC No. 36 | 0/100/0 |
| AP cC No. 37 | 36/64/0 |
| AP cC No. 38 | 0/100/0 |
| AP cC No. 39 | 17/83/0 |
| AP cC No. 40 | 1/99/0 |
| AP cC No. 41 | 9/91/0 |
| AP cC No. 42 | 25/75/0 |

As shown in the following Tables 4-6, lily varieties Acapulco, R094-19, Tiara and MP8 were transformed using Agrobacterium containing pNARI-cTr3, and 123, 46, 33 and 2 cultivars of genetically modified plants were respectively acquired. The average delphinidin content in the petioles where anthocyanins had accumulated was 10%, 15%, 20% and 4%, respectively, and the maximum delphinidin content was 63% (individual no. AP cTr3 No. 80), 48%, 54% and 7%, respectively. The analysis results are summarized in the following Tables 4 to 6. When the genetically modified Acapulco varieties were allowed to bloom and analyzed for anthocyanidins in the petals thereof, the delphinidin content in a cultivar designated as No. 80-1 was 73%. The flower color thereof was similar to that of genetically modified Acapulco (described in Example 1) that demonstrated a delphinidin content of 80%.

TABLE 4

| Individual No. | HPLC Petiole (Del/Cya/Pel: %) |
|---|---|
| AP cTr3 No. 1 | 1/99/0 |
| AP cTr3 No. 2 | 21/75/4 |
| AP cTr3 No. 3 | 0/100/0 |
| AP cTr3 No. 4 | 3/97/0 |
| AP cTr3 No. 5 | 1/99/0 |
| AP cTr3 No. 6 | 8/91/1 |
| AP cTr3 No. 7 | 30/58/12 |
| AP cTr3 No. 8 | 29/71/0 |
| AP cTr3 No. 9 | 30/67/3 |
| AP cTr3 No. 10 | 0/100/0 |
| AP cTr3 No. 11 | 18/79/3 |
| AP cTr3 No. 12 | 5/94/1 |
| AP cTr3 No. 13 | 7/93/0 |
| AP cTr3 No. 14 | 12/86/2 |
| AP cTr3 No. 15 | 18/78/4 |
| AP cTr3 No. 16 | 6/94/0 |
| AP cTr3 No. 17 | 0/100/0 |
| AP cTr3 No. 18 | 15/85/0 |
| AP cTr3 No. 19 | 39/38/23 |
| AP cTr3 No. 20 | 1/99/0 |
| AP cTr3 No. 21 | 1/99/0 |
| AP cTr3 No. 22 | 9/91/0 |
| AP cTr3 No. 23 | 23/75/2 |
| AP cTr3 No. 24 | 1/99/0 |
| AP cTr3 No. 25 | 2/98/0 |
| AP cTr3 No. 26 | 43/56/1 |
| AP cTr3 No. 27 | 31/66/3 |
| AP cTr3 No. 28 | 1/99/0 |
| AP cTr3 No. 29 | 1/99/0 |
| AP cTr3 No. 30 | 1/99/0 |
| AP cTr3 No. 31 | 1/99/0 |
| AP cTr3 No. 32 | 23/75/2 |
| AP cTr3 No. 33 | 2/97/1 |
| AP cTr3 No. 34 | 14/84/2 |
| AP cTr3 No. 35 | 1/99/0 |
| AP cTr3 No. 36 | 1/99/0 |
| AP cTr3 No. 37 | 1/99/1 |
| AP cTr3 No. 38 | 1/99/0 |

TABLE 5

| Individual No. | HPLC Petiole (Del/Cya/Pel: %) |
|---|---|
| AP cTr3 No. 39 | 0/100/0 |
| AP cTr3 No. 40 | 15/85/0 |
| AP cTr3 No. 41 | 4/96/0 |
| AP cTr3 No. 42 | 3/97/0 |
| AP cTr3 No. 43 | 22/76/2 |
| AP cTr3 No. 44 | 2/98/0 |
| AP cTr3 No. 45 | 1/99/0 |
| AP cTr3 No. 46 | 10/78/12 |
| AP cTr3 No. 47 | 7/93/0 |
| AP cTr3 No. 48 | 3/97/0 |
| AP cTr3 No. 49 | 3/97/0 |
| AP cTr3 No. 50 | 5/95/0 |
| AP cTr3 No. 51 | 7/93/0 |
| AP cTr3 No. 52 | 10/90/0 |
| AP cTr3 No. 53 | 9/88/3 |
| AP cTr3 No. 54 | 9/91/0 |
| AP cTr3 No. 55 | 1/99/0 |
| AP cTr3 No. 56 | 28/72/0 |
| AP cTr3 No. 57 | 10/90/0 |
| AP cTr3 No. 58 | 0/100/0 |
| AP cTr3 No. 59 | 27/73/0 |
| AP cTr3 No. 60 | 3/97/0 |
| AP cTr3 No. 61 | 1/99/0 |
| AP cTr3 No. 62 | 20/80/0 |
| AP cTr3 No. 63 | 1/99/0 |
| AP cTr3 No. 64 | 39/55/6 |
| AP cTr3 No. 65 | 1/99/0 |
| AP cTr3 No. 66 | 6/94/0 |
| AP cTr3 No. 67 | 0/100/0 |
| AP cTr3 No. 68 | 6/92/2 |
| AP cTr3 No. 69 | 10/88/2 |
| AP cTr3 No. 70 | 9/91/0 |
| AP cTr3 No. 71 | 0/100/0 |
| AP cTr3 No. 72 | 3/97/0 |
| AP cTr3 No. 73 | 4/93/3 |
| AP cTr3 No. 74 | 1/98/1 |
| AP cTr3 No. 75 | 1/99/0 |
| AP cTr3 No. 76 | 0/100/0 |
| AP cTr3 No. 77 | 1/99/0 |
| AP cTr3 No. 78 | 18/82/0 |
| AP cTr3 No. 79 | 13/87/0 |
| AP cTr3 No. 80 | 63/31/6 |
| AP cTr3 No. 81 | 15/85/0 |
| AP cTr3 No. 82 | 0/100/0 |

TABLE 6

| Individual No. | HPLC Petiole (Del/Cya/Pel: %) | Del |
|---|---|---|
| AP cTr3 No. 83 | 7/93/0 | 7 |
| AP cTr3 No. 84 | 5/95/0 | 5 |
| AP cTr3 No. 85 | 16/84/0 | 16 |
| AP cTr3 No. 86 | 31/67/2 | 31 |
| AP cTr3 No. 87 | 8/92/0 | 8 |
| AP cTr3 No. 88 | 1/99/0 | 1 |
| AP cTr3 No. 89 | 19/81/0 | 19 |
| AP cTr3 No. 90 | 0/100/0 | 0 |
| AP cTr3 No. 91 | 0/100/0 | 0 |
| AP cTr3 No. 92 | 36/57/7 | 36 |
| AP cTr3 No. 93 | 49/20/31 | 49 |
| AP cTr3 No. 94 | 4/93/3 | 4 |
| AP cTr3 No. 95 | 1/99/0 | 1 |
| AP cTr3 No. 96 | 3/97/0 | 3 |
| AP cTr3 No. 97 | 0/100/0 | 0 |
| AP cTr3 No. 98 | 25/65/10 | 25 |
| AP cTr3 No. 99 | 1/99/0 | 1 |
| AP cTr3 No. 100 | 6/94/0 | 6 |
| AP cTr3 No. 101 | 17/83/0 | 17 |
| AP cTr3 No. 102 | 1/98/1 | 1 |
| AP cTr3 No. 103 | 1/99/0 | 1 |
| AP cTr3 No. 104 | 20/80/0 | 20 |
| AP cTr3 No. 105 | 1/99/0 | 1 |
| AP cTr3 No. 106 | 8/92/0 | 8 |
| AP cTr3 No. 107 | 0/100/0 | 0 |
| AP cTr3 No. 108 | 2/98/0 | 2 |
| AP cTr3 No. 109 | 0/100/0 | 0 |
| AP cTr3 No. 110 | 2/98/0 | 2 |
| AP cTr3 No. 111 | 13/87/0 | 13 |
| AP cTr3 No. 112 | 45/54/1 | 45 |
| AP cTr3 No. 113 | 54/41/5 | 54 |
| AP cTr3 No. 114 | 2/98/0 | 2 |
| AP cTr3 No. 115 | 16/84/0 | 16 |
| AP cTr3 No. 116 | 2/98/0 | 2 |
| AP cTr3 No. 117 | 1/99/0 | 1 |
| AP cTr3 No. 118 | 0/100/0 | 0 |
| AP cTr3 No. 119 | 0/100/0 | 0 |
| AP cTr3 No. 120 | 25/72/3 | 25 |
| AP cTr3 No. 121 | 1/99/0 | 1 |
| AP cTr3 No. 122 | 16/84/0 | 16 |
| AP cTr3 No. 123 | 5/95/0 | 5 |
| Average | | 10.05 |

### Example 3

### Tr3M Construction and Lily Transformation

The terminator of campanula F3′5′H gene contained in the T-DNA of pNARI-Tr3 was changed from the terminator derived from nopaline synthase gene to a terminator derived from manopine synthase gene (Patent Document 1). The resulting vector was designated as pTr3M. Lily varieties Acapulco, R094-19, Tiara and MP8 were transformed using Agrobacterium containing this vector, and 6, 19, 71 and 11 cultivars, respectively, of genetically modified plants were acquired. The average delphinidin content of petioles where anthocyanins had accumulated was 38%, 34%, 7% and 22%, respectively, and the maximum delphinidin content was 72%, 70%, 54% and 65%, respectively.

### Example 4

### Tr7 Construction and Lily Transformation

(1) Acquisition of Dihydroflavonol 4-Reductase (DFR) Gene from Lilies

A cDNA library derived from the petals of lily variety Aktiva (Yamaki Noen Co., Ltd.) was prepared by digesting Lambda ZAP II vector with EcoRI and XhoI, and using the dephosphorylated Uni-ZAP XR vector for directional cloning (Agilent Technologies Inc.). The method described in WO 01/092509, for example, can be used to prepare the cDNA library. A plasmid pSPB3165 containing lily DFR cDNA (SEQ ID NO: 13) between the EcoRI and XhoI restriction enzyme sites of pBluescriptII vector was acquired by screening the resulting cDNA library with labeled geranium DFR cDNA (GenBank Accession No. AB534774, described in Plant Biotechnology (2010) 27, 375-383). Screening of the cDNA library can be carried out according to, for example, the method described in WO 81/092509.

(2) Acquisition of Lily F3′H cDNA

The aforementioned lily petal cDNA library was screened using the F3′H DNA fragment contained in pCR2.1-AP24-1 described in Example 1. The plasmid containing F3′H cDNA was designated as pSBP3175. Since this cDNA was not full length, the missing sequence on the 5′-side was amplified using the Gene Racer Kit (Invitrogen Corp.) according to the method recommended by the manufacturer. The synthetic oligonucleotide LiRG-R3: 5′-TGCTCCGCTATCAACTTATC-3′ (SEQ ID NO: 15) was used for the PCR reaction. The resulting DNA fragment was cloned in pCR-TOPOII to obtain plasmid pSPB3194. An approximately 700 bp DNA fragment obtained from this plasmid by digesting with PstI and SspI was inserted between the PstI and SspI sites of plasmid pSPB3175 to obtain plasmid pSPB3198. The synthesized lily F3′H cDNA (SEQ ID NO: 16) is contained in this plasmid.

### (3) Vector Construction

A binary vector Tr7 for suppressing expression of lily F3′H gene and DFR gene and expressing campanula F3′5′H gene and iris DFR gene in lilies was constructed.

A cassette for suppressing expression of lily DFR gene was constructed in the manner described below. An approximately 0.5 kp DNA fragment obtained by digesting plasmid pSPB3165 with EcoRI and PstI (containing a sequence encoding the vicinity of the N-terminal lily DFR cDNA) was cloned between the EcoRI and PstI sites of the plasmid pBluescriptSK—(Agilent Technologies Inc.). Plasmid pSPB3168 was obtained by ligating an approximately 0.5 kb DNA fragment obtained by digesting this plasmid with BamHI and SalI (containing a sequence encoding the vicinity of the N-terminal of lily DFR cDNA), an approximately 3.5 kb DNA fragment obtained by digesting plasmid pSPB906 (described in WO 2005/017147) with BamHI and EcoRI, and an approximately 300 bp DNA fragment obtained by digesting plasmid pSPB176 with SalI and EcoRI (containing a nopaline synthase terminator). Plasmid pSPB3178 was obtained by ligating an approximately 0.8 kb DNA fragment obtained by digesting the aforementioned plasmid pSPB3165 with BamHI and EcoRV (containing a sequence encoding a portion on the N-terminal side of lily DFR cDNA) with plasmid pSPB3168 digested with BamHI and SmaI. This plasmid contains a cassette for allowing 35S promoter to which an enhancer has been added to constitutively transcribe double-stranded RNA of lily DFR cDNA. A plasmid in which the 35S promoter to which an enhancer of plasmid pSPB3178 had been added was replaced with a 35S promoter sequence was designated as pSPB3456.

A plasmid in which a 35S promoter sequence to which an enhancer of plasmid pSPB912 (described in WO 2005/017147) had been added was replaced with 35S promoter sequence was designated as plasmid pSPB3419. This plasmid is used for the purpose of constitutively expressing iris DFR cDNA. A cassette for suppressing expression of lily F3′H gene was prepared in the manner described below. After digesting pSPB3198 with HaeII, it was further digested with BamHI followed by recovery of a DNA fragment containing the 5′-side of F3′H cDNA. Next, pSPB3194 was digested with HaeII followed by blunting, further digesting with XhoI and recovering a DNA fragment containing the 5′-side of F3′H cDNA. These DNA fragments were ligated with pSPB912 digested with BamHI and SalI to obtain plasmid pSPB3200. A plasmid in which a 35S promoter sequence to which an enhancer of pSBP3200 had been added was replaced with 35S promoter sequence was designated as plasmid pSPB3455. Transcription cassettes were respectively recovered from pSPB3456, pSPB3419 and pSPB3455 and used to construct Tr7.

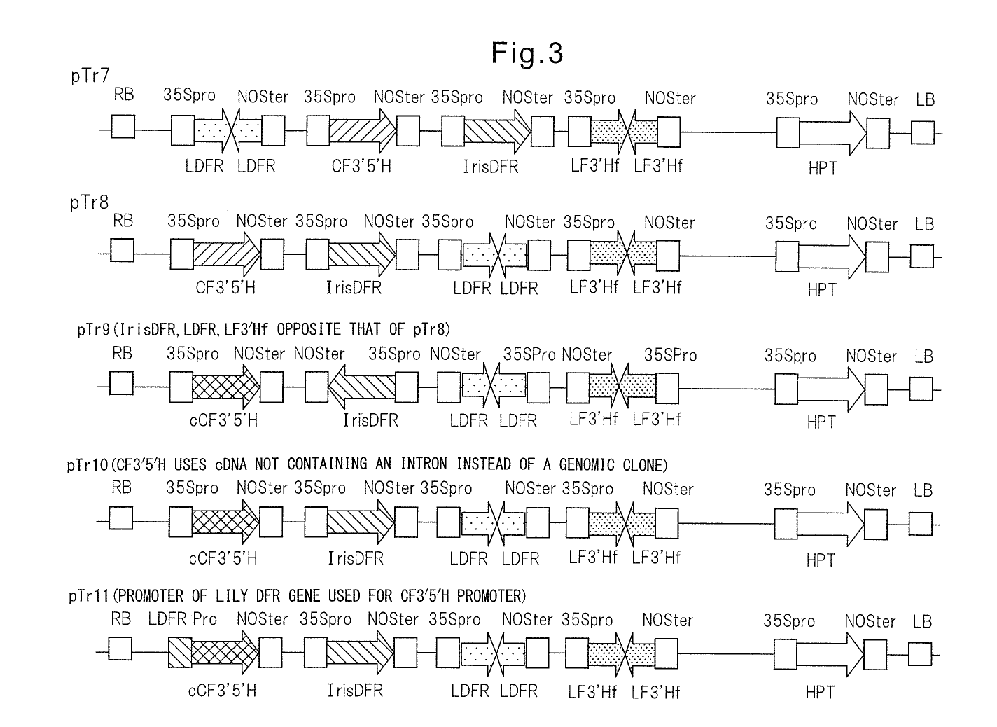

Tr7 contains five transcription cassettes on T-DNA consisting of, when moving from the right border sequence side, (i) 35S promoter sequence, sequence for transcribing double-stranded RNA of lily DFR cDNA and nopaline synthase terminator, (ii) 35S promoter sequence, campanula F3′5′H gene sequence containing an intron, and nopaline synthase terminator (same as that contained in Tr3), (iii) 35S promoter sequence, iris DFR gene sequence and nopaline synthase terminator, (iv) 35S promoter sequence, sequence for transcribing double-stranded RNA of lily F3′H cDNA and nopaline synthase terminator (same as contained in Tr3), and (v) 35S promoter sequence, hygromycin phosphotransferase gene sequence, and nopaline synthase terminator (same as contained in Tr3). A map of Tr7 is shown in FIG. 3.

### (4) Preparation of Genetically Modified Lilies

Lily varieties Acapulco, R094-19, Tiara and MP8 were transformed using Agrobacterium containing Tr7, and 22, 80, 32 and 11 cultivars, respectively, of genetically modified plants were acquired. The average delphinidin content of petioles in which anthocyanins accumulated was 15%, 13%, 6% and 28%, respectively, and the maximum delphinidin content was 78%, 88%, 56% and 87%, respectively.

The R094 genetically modified cultivar R094-19 Tr7 No. 52-3 had a petal delphinidin content of 99%.

### Example 5

### Tr8 Construction and Lily Transformation

Binary vector Tr8 was prepared in which the order of the expression cassettes on T-DNA differs from that of binary vector Tr7. Tr8 contains five transcription cassettes on T-DNA consisting of, when moving from the right border sequence side, (i) 35S promoter sequence, campanula F3′5′H gene sequence containing an intron, and nopaline synthase terminator (same as that contained in Tr3), (ii) 35S promoter sequence, iris DFR gene sequence and nopaline synthase terminator, (iii) 35S promoter sequence, sequence for transcribing double-stranded RNA of lily DFR cDNA, and nopaline synthase terminator (same as that contained in Tr7), (iv) 35S promoter sequence, sequence for transcribing double-stranded RNA of lily F3′H cDNA and nopaline synthase terminator (same as contained in Tr7), and (v) 35S promoter sequence, hygromycin phosphotransferase gene sequence, and nopaline synthase terminator (same as contained in Tr3). A map of Tr8 is shown in FIG. 3. Lily varieties Acapulco, R094-19 and Tiara were transformed using Agrobacterium containing Tr8, and 66, 6 and 87 cultivars, respectively, of genetically modified plants were acquired. The average delphinidin content of petioles in which anthocyanins accumulated was 15%, 6% and 30%, respectively (the petioles of 8 of the 87 cultivars were analyzed), and the maximum delphinidin content was 51%, 6% and 70%, respectively.

### Example 6

### Tr9 Construction and Lily Transformation

Binary vector Tr9 was constructed that expresses campanula F3′5′H cDNA. A map of Tr9 is shown in FIG. 3. Tr9 contains five transcription cassettes on T-DNA consisting of, when moving from the right border sequence side, (i) nopaline synthase terminator (same as that contained in cTr3), cDNA sequence of campanula F3′5′H gene, and 35S promoter sequence, (ii) 35S promoter sequence, sequence for transcribing double-stranded RNA of lily DFR cDNA, and nopaline synthase terminator (same as that contained in Tr7 but having a different orientation), (iii) nopaline synthase terminator, sequence for transcribing double-stranded RNA of lily F3′H cDNA, and 35S promoter sequence (same as that contained in Tr7 but having a different orientation), (iv) nopaline synthase terminator, sequence for transcribing double-stranded RNA of lily F3′H cDNA (same as that contained in Tr7 but having a different orientation), and (v) 35S promoter sequence, hygromycin phosphotransferase gene sequence, and nopaline synthase terminator (same as contained in Tr3). Lily varieties Acapulco, R094-19 and Tiara were transformed using Agrobacterium containing Tr9, and 8, 51 and 33 cultivars, respectively, of genetically modified plants were acquired. The average delphinidin content of petioles in which anthocyanins accumulated was 2%, 12% (the petioles of 44 of the 51 cultivars were analyzed) and 13%, respectively, and the maximum delphinidin content was 9%, 68% and 38%, respectively.

### Example 7

### Construction of Tr10 and Lily Transformation

Tr10 contains five transcription cassettes on T-DNA consisting of, when moving from the right border sequence side, (i) 35S promoter sequence, cDNA sequence of campanula F3′5′H gene, and nopaline synthase terminator (same as that contained in cTr3), (ii) 35S promoter sequence, iris DFR gene sequence, and nopaline synthase terminator (same as that contained in Tr7), (iii) 35S promoter sequence, sequence for transcribing double-stranded RNA of lily DFR cDNA, and nopaline synthase terminator (same as that contained in Tr7), (iv) 35S promoter sequence, sequence for transcribing double-stranded RNA of lily F3′H cDNA, and nopaline synthase terminator (same as that contained in Tr7), and (v) 35S promoter sequence, hygromycin phosphotransferase gene sequence, and nopaline synthase terminator (same as contained in Tr3). A map of Tr10 is shown in FIG. 3. Lily varieties Acapulco, R094-19, Tiara and MP8 were transformed using Agrobacterium containing Tr10, and 41, 14, 78 and 10 cultivars, respectively, of genetically modified plants were acquired. The average delphinidin content of petioles in which anthocyanins accumulated was 23%, 26%, 15% (the petioles of 19 cultivars were analyzed) and 36%, respectively, and the maximum delphinidin content was 80%, 87%, 75% and 74%, respectively.

The flower color of the genetically modified Acapulco cultivar AP Tr10 No. 34-1 changed distinctly (color chart no. N78A), and the delphinidin content in the petals was 82%.

### Example 8

### Construction of Tr11 and Lily Transformation

### (1) Isolation of Promoter Sequence of Lily DFR Gene

Isolation of the promoter sequence of lily DFR gene was carried out by inverse PCR. Four primers consisting of cLD2L: 5′-AGGGTCTCTCACAGTAGCCCGAACCGTGTAGCCA-3′ (SEQ ID NO: 18), LD1L: 5′-ACTAGATCTTCCTGGAGCTGATGAACGGCTAACC-3′ (SEQ ID NO: 19), cLD1L: 5′-AACATAGCCACTAGCTCCAGTAACCACCACGGGTC-3′ (SEQ ID NO: 20) and LD3L: 5′-AGCAAATCCAGCTTGTTCACTTGGATGACGTATGC-3′ (SEQ ID NO: 21) were synthesized based on the DFR cDNA sequence of the lily variety Montreux (pink flowers) registered in the GenBank DNA database (DNA Database Accession No. AB058641). DNA was purified from the leaves of the lily variety Montreux (Yamaki Noen Co., Ltd.) using the DNeasy Plant Mini Kit (Qiagen Corp.). After cleaving the resulting DNA with restriction enzyme EcoRI, cyclic DNA was prepared by a self-ligation reaction of the DNA fragments using a DNA Ligation Kit (Mighty Mix, Takara Bio Inc.). A first round of a PCR reaction was carried out using the Takara LA Taq Hot Start Version (Takara Bio Inc.) by using this cyclic DNA as template and using cLD2L and LD1L as primers. Moreover, a second round of a PCR reaction was carried out using the Takara LA Taq Hot Start Version by using the amplified DNA as template and using cLD1L and LD3L as primers to obtain a DNA fragment containing the promoter portion of DFR. The resulting DNA fragment was ligated to plasmid pCR2.1 (Invitrogen Corp.). The resulting plasmid was designated as pCR-LDE. Moreover, the promoter sequence of lily DFR gene (SEQ ID NO: 24) was amplified using this plasmid as template, and using the synthetic nucleotides AscI-HindIII-LDE6-F (5′-GGCGCGCCAAGCTTGAATTCTTAGCGGAAATTATCC-3′ (SEQ ID NO: 22) and XbaI-LDE6-R (5′-TCTAGAATCTCTCTCTCTCCCCCTC-3′ (SEQ ID NO: 23) as primers. A DNA fragment containing this sequence can be recovered by digesting with HindIII and XbaI.

### (2) Vector Construction and Lily Transformation

Tr11 contains five transcription cassettes on T-DNA consisting of, when moving from the right border sequence side, (i) lily DFR promoter sequence, campanula F3′5′H gene cDNA sequence, and nopaline synthase terminator, (ii) 35S promoter sequence, iris DFR gene sequence, and nopaline synthase terminator (same as that contained in Tr7), (iii) 35S promoter sequence, sequence for transcribing double-stranded RNA of lily DFR cDNA, and nopaline synthase terminator (same as that contained in Tr7), (iv) 35S promoter sequence, sequence for transcribing double-stranded RNA of lily F3′H cDNA, and nopaline synthase terminator (same as that contained in Tr7), and (v) 35S promoter sequence, hygromycin phosphotransferase gene sequence, and nopaline synthase terminator (same as contained in Tr3). A map of Tr11 is shown in FIG. 3. Lily varieties R094-19, Tiara and MP8 were transformed using Agrobacterium containing Tr11, and 66, 120 and 18 cultivars, respectively, of genetically modified plants were acquired. The average delphinidin content of petioles in which anthocyanins accumulated was 40%, 14% and 38%, respectively, and the maximum delphinidin content was 100% (52 cultivars analyzed), 54% (14 cultivars analyzed) and 92% (11 cultivars analyzed), respectively.

### INDUSTRIAL APPLICABILITY

According to the present invention, delphinidin not found in nature can be produced and lilies in which flower color has changed to blue can be produced by suppressing expression of one or multiple endogenous flavonoid synthase genes in lilies, and expressing F3′S′H gene or F3′5′H gene and DFR gene. Thus, the present invention can be preferably used in such industries as the flowering plant industry.

### BRIEF DESCRIPTION OF THE REFERENCE SYMBOLS

RB Right border
LB Left border
35Spro Cauliflower mosaic virus 35S promoter
NOSter Nopaline synthase terminator
LF3′hf Lily F3′H gene fragment
CF3′5′H Campanula F3′5′H gene
IrisDFR Iris DFR gene
cCF3′5′H Campanula F3′5′H cDNA
LDFR Lily DFR
HPT Hygromycin resistance gene

1. A method for producing lilies containing delphinidin in the petals thereof, comprising the following steps:
introducing into lilies a campanula-derived flavonoid 3′,5′-hydroxylase (F3′5′H) gene composed of a nucleotide sequence represented by SEQ ID NO: 1 or SEQ ID NO: 11, or a nucleotide sequence having at least 90% sequence identity with the nucleotide sequence represented by SEQ ID NO: 1 or SEQ ID NO: 11 that also encodes a polypeptide having F3′5′H activity, introducing a fragment of lily-derived flavonoid 3′-hydroxylase (F3′H) gene composed of a nucleotide sequence represented by SEQ ID NO: 3 or SEQ ID NO: 16, or a nucleotide sequence having at least 90% sequence identity with the nucleotide sequence represented by SEQ ID NO: 3 or SEQ ID NO: 16 that also encodes a polypeptide having F3′H activity, and synthesizing delphinidin by the action of the introduced F3′5′H gene while suppressing expression of endogenous F3′H gene that acts on cyanidin synthesis in lily petals.

2. The method according to claim 1, wherein the suppression of expression of endogenous F3′H is carried out in the RNAi method.

3. The method according to claim 1, wherein the campanula-derived F3′5′H gene is composed of a nucleotide sequence having at least 95% sequence identity with the nucleotide sequence represented by SEQ ID NO: 1 or SEQ ID NO: 11 that also encodes a polypeptide having F3′5′H activity.

4. The method according to claim 3, wherein the campanula-derived F3′5′H gene is composed of a nucleotide sequence represented by SEQ ID NO: 1 or SEQ ID NO: 11.

5. The method according to claim 1, wherein the fragment of lily-derived F3′H gene is composed of a nucleotide sequence having at least 95% sequence identity with the nucleotide sequence represented by SEQ ID NO: 3 or SEQ ID NO: 16 that also encodes a polypeptide having F3′H activity.

6. The method according to claim 5, wherein the fragment of lily-derived F3′H gene is composed of a nucleotide sequence represented by SEQ ID NO: 3 or SEQ ID NO: 16.

7. The method according to claim 1, further comprising: introducing a fragment of lily-derived dihydroflavonol 4-reductase (DFR) gene composed of a nucleotide sequence represented by SEQ ID NO: 13 or a nucleotide sequence having at least 90% sequence identity with the nucleotide sequence represented by SEQ ID NO: 13 that also encodes a polypeptide having DFR activity, suppressing expression of endogenous DFR that acts on cyanidin synthesis in lily petals, and simultaneously introducing an iris-derived foreign DFR gene.

8. The method according to claim 7, wherein the suppression of expression of endogenous DFR is carried out in the RNAi method.

9. The method according to claim 1, further comprising: using a lily DFR promoter sequence represented by SEQ ID NO: 24.

10. A lily, tissue thereof, portion thereof, vegetatively propagated plant thereof or progeny thereof, which contains delphinidin in the petals thereof produced according to the method according to claim 1.

11. The lily, tissue thereof, portion thereof, vegetatively propagated plant thereof or progeny thereof according to claim 10, wherein the tissue or portion thereof is a cut flower.

12. The method according to claim 2, wherein the campanula-derived F3′5′H gene is composed of a nucleotide sequence having at least 95% sequence identity with the nucleotide sequence represented by SEQ ID NO: 1 or SEQ ID NO: 11 that also encodes a polypeptide having F3′5′H activity.

13. The method according to claim 2, wherein the fragment of lily-derived F3′H gene is composed of a nucleotide sequence having at least 95% sequence identity with the nucleotide sequence represented by SEQ ID NO: 3 or SEQ ID NO: 16 that also encodes a polypeptide having F3′H activity.

14. The method according to claim 2, further comprising: introducing a fragment of lily-derived dihydroflavonol 4-reductase (DFR) gene composed of a nuceotide sequence represented by SEQ ID NO: 13 or a nucleotide sequence having at least 90% sequence identity with the nucleotide sequence represented by SEQ ID NO: 13 that also encodes a polypeptide having DFR activity, suppressing expression of endogenous DFR that acts on cyanidin synthesis in lily petals, and simultaneously introducing an iris-derived foreign DFR gene.

15. The method according to claim 2, further comprising: using a lily DFR promoter sequence represented by SEQ ID NO: 24.

16. A lily, tissue thereof, portion thereof, vegetatively propagated plant thereof or progeny thereof, which contains delphinidin in the petals thereof produced according to the method according to claim 2.

17. A lily, tissue thereof, portion thereof, vegetatively propagated plant thereof or progeny thereof, which contains delphinidin in the petals thereof produced according to the method according to claim 4.

18. A lily, tissue thereof, portion thereof, vegetatively propagated plant thereof or progeny thereof, which contains delphinidin in the petals thereof produced according to the method according to claim 6.

19. A lily, tissue thereof, portion thereof, vegetatively propagated plant thereof or progeny thereof, which contains delphinidin in the petals thereof produced according to the method according to claim 8.
